(19) United States
(12) Patent Application Publication
(10) Pub. No.: US 2017/0029515 A1
(43) Pub. Date: Feb. 2, 2017

(54) METHOD OF INHIBITION OF LEUKEMIC STEM CELLS

(71) Applicants: CSL Limited, Perkville, VI (US); University Health Network, Toronto (CA)

(72) Inventors: John Edgar Dick, Toronto (CA); Liqing JIN, Toronto (CA); Gino Luigi VAIRO, Northcote (AU); David Paul GEARING, Camberwell (AU); Samantha Jane BUSFIELD, Brunswick (AU)

(73) Assignees: CSL Limited, Parkville (AU); University Health Network, Toronto (CA)

(21) Appl. No.: 15/233,260

(22) Filed: Aug. 10, 2016

Related U.S. Application Data
(63) Continuation of application No. 14/552,789, filed on Nov. 25, 2014.
Application No. 14/552,789 is a continuation of application No. 13/693,326, filed on Dec. 4, 2012, now abandoned.
Application No. 13/693,326 is a continuation of application No. 12/745,607, filed on Oct. 22, 2010, now abandoned.
(60) Provisional application No. 60/996,819, filed on Dec. 6, 2007.

(51) Int. Cl. C07K 16/28; C07K 16/30; A61K 31/7068; A61K 45/06; A61K 39/395

(52) U.S. Cl. CPC C07K 16/2866; A61K 45/06; A61K 39/3955; A61K 31/7068; C07K 16/3061; C07K 2317/41; C07K 2317/24; C07K 2317/21; C07K 2317/567

(57) ABSTRACT

A method for inhibition of leukemic stem cells expressing IL-3Rα (CD123), comprises contacting the cells with an antigen binding molecule comprising a Fc region or a modified Fc region having enhanced Fc effector function, wherein the antigen binding molecule binds selectively to IL-3Rα (CD123). The invention includes the treatment of a hematologic cancer condition in a patient by administration to the patient of an effective amount of the antigen binding molecule.

### FIELD OF THE INVENTION

This invention relates to a method for the inhibition of leukemic stem cells, and in particular for the inhibition of leukemic stem cells associated with acute myelogenous leukemia (AML) and other haematologic cancer conditions as an effective therapy against these hematologic cancer conditions.

### BACKGROUND OF THE INVENTION

Hematological cancer conditions are the types of cancer such as leukemia and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Leukemic stem cells (LSCs) are cancer cells that possess characteristics associated with normal stem cells, that is, the property of self renewal and the capability to develop multiple lineages. Such cells are proposed to persist in hematological cancers such as AML as distinct populations.¹

Acute myelogenous leukemia (AML) is a clonal disorder clinically presenting as increased proliferation of heterogeneous and undifferentiated myeloid blasts. The leukemic hierarchy is maintained by a small population of LSCs, which have the distinct ability for self-renewal, and are able to differentiate into leukemic progenitors¹. These progenitors generate the large numbers of leukemic blasts readily detectable in patients at diagnosis and relapse, leading ultimately to mortality²⁻⁴. AML-LSC have been commonly reported as quiescent cells, in contrast to rapidly dividing clonogenic progenitors^{3,5,6}. This property of LSCs renders conventional chemotherapeutics that target proliferating cells less effective, potentially explaining the current experience in which a high proportion of AML patients enter complete remission, but almost invariably relapse, with <30% of adults surviving for more than 4 years⁷. In addition, minimal residual disease occurrence and poor survival has been attributed to high LSC frequency at diagnosis in AML patients⁸. Consequently, it is imperative for the long term management of AML (and similarly other above mentioned hematological cancer conditions) that new treatments are developed to specifically eliminate LSCs⁹⁻¹⁴.

AML-LSCs and normal hematopoietic stem cells (HSCs) share the common properties of slow division, self-renewal ability, and surface markers such as the CD34⁺CD38⁻ phenotype. Nevertheless, LSCs have been reported to possess enhanced self-renewal activity, in addition to altered expression of other cell surface markers, both of which present targets for therapeutic exploitation. Interleukin-3 (IL-3) mediates its action through interaction with cell surface receptors that consist of 2 subunits, the a subunit (CD123) and the β common (β_{c}) chain (CD131). The interaction of an α chain with a β chain forms a high affinity receptor for IL-3, and the β_{c} chain mediates the subsequent signal transduction^{15,16}. Over-expression of CD123 on AML blasts, CD34⁺ leukemic progenitors and LSCs relative to normal hematopoietic cells has been widely reported¹⁷⁻²³, and has been proposed as a marker of LSCs in some studies^{24,25}. CD131 was also reported to be expressed on AML cells^{21,25} but there are conflicting reports on its expression on AML-LSCs^{23,25}.

Overexpression of CD123 on AML cells confers a range of growth advantages over normal hematopoietic cells, with a large proportion of AML blasts reported to proliferate in culture in response to IL-3²⁶⁻³¹. Moreover, high-level CD123 expression on AML cells has been correlated with: the level of IL-3-stimulated STAT-5 activation; the proportion of cycling cells; more primitive cell surface phenotypes; and resistance to apoptosis. Clinically, high CD123 expression in AML is associated with lower survival duration, a lower complete remission rate and higher blast counts at diagnosis^{19,21,32}.

The increased expression of CD123 on LSCs compared with HSCs presents an opportunity for therapeutic targeting of AML-LSCs. The monoclonal antibody (MAb) 7G3, raised against CD123, has previously been shown to inhibit IL-3 mediated proliferation and activation of both leukemic cell lines and primary cells³³. However, it has remained unclear whether targeting CD123 can functionally impair AML-LSCs, and whether it can inhibit the homing, lodgment and proliferation of AML-LSCs in their bone marrow niche. Moreover, the relative contributions of direct inhibition of IL-3 mediated signaling versus antibody-dependent cell-mediated cytotoxicity (ADCC) in the ability of 7G3 to target AML-LSCs remain unresolved.

U.S. Pat. No. 6,177,078 (Lopez) discloses the anti-IL-3Receptor alpha chain (IL-3Rα) monoclonal antibody 7G3, and the ability of 7G3 to bind to the N-terminal domain, specifically amino acid residues 19-49, of IL-3Rα. Accordingly, this patent discloses the use of a monoclonal antibody such as 7G3 or antibody fragment thereof with binding specificity for amino acid residues 19-49 of IL-3Rα in the treatment of conditions resulting from an overproduction of IL-3 in a patient (including myeloid leukemias, lymphomas and allergies) by antagonizing the functions of the IL-3.

U.S. Pat. No. 6,733,743 (Jordan) discloses a method of impairing a hematologic cancer progenitor cell that expresses CD123 but does not significantly express CD131, by contacting the cell with a composition of an antibody and a cytotoxic agent (selected from a chemotherapeutic agent, a toxin or an alpha-emitting radioisotope) whereby the composition binds selectively to CD123 in an amount effective to cause cell death. The hematologic cancer may be leukemia or a malignant lymphoproliferative disorder such as lymphoma.

In work leading to the present invention, the inventors have tested the ability of MAb 7G3 to exploit the overt differences in CD123 expression and function between AML-LSCs and HSCs. MAb 7G3 inhibited the IL-3 signaling pathway and proliferation of primary AML cells. Moreover, the homing and engraftment of AML blasts in the nonobese diabetic/severe combined immunodeficient (NOD/SCID) xenograft model were profoundly reduced by MAb 7G3, and LSC function was inhibited.

### SUMMARY OF THE INVENTION

In one aspect, the present invention provides a method for inhibition of leukemic stem cells expressing IL-3Rα (CD123), which comprises contacting said cells with an antigen binding molecule comprising a Fc region or a modified Fc region having enhanced Fc effector function, wherein said antigen binding molecule binds selectively to IL-3Rα (CD123).

The present invention also provides a method for the treatment of a hematologic cancer condition in a patient, which comprises administration to the patient of an effective amount of an antigen binding molecule comprising a Fc region or a modified Fc region having enhanced Fc effector function, wherein said antigen binding molecule binds selectively to IL-3Rα (CD123).

In another aspect, the present invention also provides the use of an antigen binding molecule comprising a Fc region or a modified Fc region having enhanced Fc effector function in, or in the manufacture of a medicament for, the inhibition of leukemic stem cells expressing IL-3Rα (CD123), wherein said antigen binding molecule binds selectively to IL-3Rα (CD123).

In this aspect, the invention also provides the use of an antigen binding molecule comprising a Fc region or a modified Fc region having enhanced Fc effector function in, or in the manufacture of a medicament for, the treatment of a hematologic cancer condition in a patient, wherein said antigen binding molecule binds selectively to IL-3Rα (CD123).

The present invention also provides an agent for inhibition of leukemic stem cells expressing IL-3Rα (CD123), which comprises an antigen binding molecule comprising a Fc region or a modified Fc region having enhanced Fc effector function, wherein said antigen binding molecule binds selectively to the IL-3Rα (CD123).

In this aspect, the invention also provides an agent for the treatment of a hematologic cancer condition in a patient, which comprises an antigen binding molecule comprising a Fc region or a modified Fc region having enhanced Fc effector function, wherein said antigen binding molecule binds selectively to IL-3Rα (CD123).

### BRIEF DESCRIPTION OF THE DRAWINGS

FIGS. 1A-1E show that MAb 7G3 inhibits IL-3-stimulated phosphorylation of CD131, and proliferation, of primary AML cells. (FIG. 1A) Primary AML cells from two individual patients were incubated with antibody at the concentrations shown in the figure for 30 min on ice. Without washing, cells were stimulated with IL-3 (1 nM for 10 min at 37° C.). Immediately following stimulation cells were lysed. Lysates were run on SDS-PAGE and immunoblotted with MAb 4G10 and then the blots were stripped and re-probed with MAb 1C1 as a loading control. (FIGS. 1B-1E) Proliferation of primary AML cells assessed by ³H-thymidine incorporation into TCA insoluble material. (FIGS. 1B-1D) Freshly isolated mononuclear cells from 3 individual AML patients were incubated with a titration of MAb 7G3 for 48 hours either in the absence (Δ, dashed line) or presence of cytokine: IL-3 at 1 ng/mL (⋄, dotted line) or GM-CSF at 0.1 ng/mL (▪, solid line). Data points show mean±s.e.m. of triplicate points. (FIG. 1E) Thawed cells from 35 patients with AML were analyzed for inhibition of proliferation by MAb 7G3 (1 μg/mL) in the absence or presence of IL-3 (1 ng/mL). Inhibition was shown in 32 of 35 patients tested. In 9 of those patients proliferation levels fell to below that in the absence of IL-3 (constitutive proliferation). Proliferation was quantified using ³H-Thymidine incorporation and liquid scintillation counting.

FIGS. 2A-2G show that CD123 neutralization inhibits homing and engraftment of primary AML cells in NOD/SCID mice. Engraftment of primary AML cells from 10 patients (FIG. 2A), or normal bone marrow (NBM) or cord blood (CB) from 5 individuals (FIG. 2B), following ex vivo exposure to 7G3 (grey bars) or IgG2a (black bars) (10 μg/mL, 2 h). Following antibody treatment cells were transplanted into sublethally irradiated NOD/SCID mice, culled at 4-8 (FIG. 2A) or 4-11 (FIG. 2B) weeks, and the proportion of human CD45⁺ cells in the femoral bone marrow estimated by flow cytometry. For each sample, 3 to mice were used per treatment group. AML-8 and AML-8-rel correspond to leukemic cells harvested from the same patient at diagnosis and relapse, respectively. NBM-4 and CB-1 originated from pooled samples. (FIG. 2C) Kaplan-Meier event-free survival curve of mice transplanted with IgG2a (n=10, solid line) or 7G3 (n=10, dotted line) ex vivo treated AML-9 cells. (FIG. 2D) Homing efficiency of IgG2a (black bars), 7G3 (grey bars) ex vivo treated AML-8-rel or AML-9 cells to the bone marrow and spleen, assessed 24 h post-transplantation. (FIG. 2E) Engraftment levels of AML-8-rel cells in mice transplanted with IgG2a (white bars) or 7G3 (black bars) ex vivo treated cells, following intravenous infusion (IV) or intrafemoral injection (IF). For the IF transplanted mice, engraftment levels in the right femur (RF) where AML cells were transplanted, and in non-transplanted bones (WBM) are shown. For (FIG. 2D) and (FIG. 2E) 4-5 mice were used per treatment group. Mice were sacrificed at 5 weeks post-transplantation. Values represent mean±s.e.m. Significant differences between control IgG2a and treated mice are indicated: *, P<0.05; **, P<0.01; ***, P≦0.0001. (FIG. 2F) Absolute number of CD34⁺38⁻ AML cells homed in the BM and spleen of NOD/SCID mice injected with ex vivo 7G3-treated leukemic cells. N=2-3 mice per group for AML-8 and n=5 mice per group for AML-9. Values represent mean±SEM. (g) Homing efficiency of sorted CD34⁺CD38⁻ AML-9 cells after ex vivo treatment into both BM and spleen of mice. N=3 mice per treatment group.

FIGS. 3A-3F show that administration of 7G3 to NOD/SCID mice reduces AML engraftment. (FIG. 3A) Engraftment levels of AML-1 cells in the femoral bone marrow of irradiated NOD/SCID mice which had received a single dose of IgG2a control or 7G3 (300 μg) 6 h prior to transplantation. Mice were culled at 5 weeks post transplantation. (FIG. 3B) Engraftment of AML-1, 2, and 3 in NOD/SCID mice treated with IgG2a (black bars) or 7G3 (grey bars). Treatments were initiated at 24 hours post-transplantation, 300 μg per dose, every other day for 4 doses. Mice were culled at 5 weeks post-transplantation. ([[c]] FIG. 3C) CD123 expression on bone marrow-derived cells, and (FIG. 3D) engraftment levels in the peripheral blood and spleen, of AML-1 cells inoculated into mice, then IgG2a or 7G3 treatments initiated 4 days post transplantation for a total of 12 injections administered 3 times/week. Mice were culled at 5 weeks post-transplantation. (FIG. 3E) Engraftment levels of AML-2 cells in the bone marrow when IgG2a (dotted line) or 7G3 (solid line) treatments were initiated 28 days post transplantation and continued 3 times/week until time of sacrifice. Between 3 and 10 mice were used per treatment group. Values represent mean±s.e.m. (FIG. 3F) Percentage of human AML-1 cells in the BM of NOD/SCID mice after 4 doses of 7G3 or IgG2a control at 300 μg/dose, administered 3 times a week starting on Day 28 post transplantation. Each individual symbol represents value obtained from a single mouse. Significant differences between IgG2a control and 7G3 treated mice are indicated: *, P<0.05; **, P<0.005.

FIGS. 4A-4D show that administration of 7G3 and Ara-C to mice with established AML disease blocks LSC repopulation of secondary recipient mice. (FIG. 4A) Engraftment levels of AML-10 cells in the bone marrow and spleen of primary mice treated with Ara-C combined with either IgG2a or 7G3 as shown in the schematic, (FIG. 4B) homing efficiency to bone marrow and spleen, (FIG. 4C) engraftment levels, and (FIG. 4D) proportion of CD34⁺38⁻ cells in the secondary graft, of leukemic cells harvested from the bone marrows of mice treated in (FIG. 4A), and transplanted into secondary recipient mice. Horizontal bars indicate the mean value. Significant differences between IgG2a plus Ara-C control and 7G3 plus Ara-C treated group are indicated: *, P<0.05 and ** P<0.01.

FIGS. 4E-4H show (FIG. 4E) engraftment levels of AML-10 cells in BM and spleen after 10 weeks of 7G3 or control IgG2a treatment. Antibody treatment was initiated at Day 28 post transplantation, 300 μg per mouse thrice weekly, as shown in the schematic overview. (FIGS. 4F-4H) Homing efficiency (FIG. 4F), levels of engraftment in the BM and spleen (FIG. 4G), and the percentage of CD34⁺CD38⁻ cells in the BM (FIG. 4H) of secondary recipient mice. Mice in C and D were analyzed at 12 weeks post transplantation. Each symbol represents a single mouse, horizontal bars indicate the mean value. *, P<0.05; **, P<0.01 between control IgG2a and 7G3 groups.

FIGS. 4I-4J show (FIG. 4I) engraftment levels of AML-9 cells in BM and spleen after 10 weeks of 7G3 or control IgG2a treatment. Antibody treatment was initiated at Day 28 post transplantation, 300 μg per mouse thrice weekly, as shown in the schematic overview. (FIG. 4J) Levels of engraftment in the BM of secondary recipient mice. Secondary mice were analyzed at 8 weeks post transplantation. Each symbol represents a single mouse, horizontal bars indicate the mean value. **, P<0.01 between control IgG2a and 7G3 groups.

FIGS. 5A-5B show that natural killer (NK) lymphocytic cells contribute to the 7G3-mediated inhibition of AML engraftment. (FIG. 5A) Level of engraftment, and (FIG. 5B) homing efficiency of AML-8-rel cells treated ex vivo with IgG2a (white bars) or 7G3 (black bars) (10 μg/mL, 2 h) and transplanted into NOD/SCID mice without (−) or with (+) prior CD122⁺ NK cell depletion. Four mice were used for each treatment group. Values represent mean±s.e.m. Significant differences are indicated: *, P<0.05 and ** P<0.01.

FIGS. 6A-6B show that MAb 7G3, but not 6H6 nor 9F5, inhibits IL-3-stimulated phosphorylation of CD131 (β_{c}), STAT-5 and Akt in IL-3 dependent cell lines and AML cells. (FIG. 6A) TF-1 cells were incubated with varying concentrations of 7G3, 9F5 or 6H6 for 30 min on ice. Without washing, cells were stimulated with IL-3 (1 nM for 10 min at 37° C.). Immediately following stimulation cells were lysed and CD131 immunoprecipitated as described in the methods. Immunoprecipitates were separated by SDS-PAGE and immunoblotted with antibodies to phosphorylated tyrosine residues (4G10), phosphorylated STAT-5 or phosphorylated Akt. Blots were stripped and re-probed with antibody to βc (1C1) as a loading control. (FIG. 6B) 7G3 inhibition of IL-3 induced activation of STAT-5 was also confirmed by intracellular FACS staining of the TF-1 and M07e cell lines, and primary AML-9 cells. Mock treatment (dotted line), IL-3 alone (10 ng/mL 2 h, solid line), IL-3 plus 7G3 (10 ng/mL, dashed line).

FIG. 7 shows that the intensity of CD123 expression on CD34⁺/CD38⁻ cells inversely correlates with the ability of 7G3 to inhibit engraftment in NOD/SCID mice. The Y-axis represents the logarithmic of RFI of CD123 expression on the CD34⁺/CD38⁻ fraction for each patient or donor specimen. The X-axis plots the logarithmic of the engraftment level of 7G3 ex vivo-treated group standardized to % of IgG2a control taken as 100% for each individual patient or donor sample. Each point represents a separate experiment reflecting the average value from 3-10 mice per treatment group and each experiment performed using different AML patient (solid symbols) or normal BM samples (open symbols). All mice were analysed after 4-6 weeks after engraftment. Each engraftment data point was based on measurements from 3-10 mice shown in FIG. 2a.

FIGS. 8A-8D show CD107a expression in NK cells with AML cells as target cells. Peripheral Blood Mononuclear cells (PBMCs) from a normal healthy donor were incubated with primary human AML cells (RMH003) at a ratio of 1:1 (FIGS. 8A & 8B), either with IgG1 control (10 μg/mL) (FIGS. 8A & 8C) or CSL360 (10 μg/mL) (FIGS. 8B & 8D) for three hours at 37° C. To assess non-specific expression of CD107a, PBMC were incubated with antibody and no target cells (1:0) (FIGS. 8C & 8D).

FIG. 9 shows a histogram plot of the data generated in the experiment depicted in FIG. 8 and as indicated also includes samples in which no antibody was added.

FIG. 10 shows homing efficiency of a AML-8-rel sample treated ex vivo with 10 μg/mL IgG2a, intact 7G3, 6H6 or 9F5 antibodies and the F(ab′)2 fragments of 7G3 (7G3 Fab) and 6H6 (6H6 Fab) prior to inoculation into NOD/SCID mice. Homing efficiency of human mononuclear cells into the bone marrow was measured after 16 hrs. For each sample, 3 mice were used per treatment group.

FIGS. 11A-11B show engraftment of primary AML cells from two patients (AML-9 and AML10) in sublethally irradiated NOD/SCID mice following ex vivo exposure to 10 μg/mL IgG2a, intact 7G3 or 9F5 antibodies and the F(ab′)2 fragments of 7G3 (7G3 Fab) and 9F5 (9F5 Fab). AML engraftment was assessed 4 weeks post inoculation as the proportion of human CD45+ cells in the femoral bone marrow estimated by flow cytometry. For each sample, 5 mice were used per treatment group.

FIG. 12 shows comparison of ADCC activities of chimeric CSL360, human CSL360 and its Fc variants. Calcein AM labeled CTLEN cells were incubated with different antibodies and freshly isolated PBMC from a normal human donor. Ratio of PBMC to CTLEN cells was 100:1. Cells were incubated for 4 hours at 37° C. in an incubator with 5% CO₂. After the incubation period, cells were centrifuged and 100 μL of supernatant transferred to a fresh plate. Fluorescence in the supernatant was measured using a Wallac microplate reader (excitation filter 485 nm, emission filter 535 nm). Antibodies used were either chimeric CSL360 (open bars), humanized CSL360 (solid bars), humanized CSL360 with two amino acid changes (diagonal lines) or humanized CSL360 with three amino acid changes (dotted). Human IgG1 (horizontal lines) and wells with no antibody (vertical lines) were included as controls.

FIGS. 13A-13B show (FIG. 13A) Biacore analysis of hCSL360, and three variants thereof, binding to FcRs. huCSL 360 and three variants thereof were individually captured on a BIAcore CM5 chip coupled with CD123. huFcγRI, huFcγRIIb/c and huFcγRIIIa, at concentrations ranging from 0.4 nM to 800 nM, were flowed over the respective surfaces and the responses used to determine KAs. Affinities are reported as fold increase over hCSL360 which is assigned a relative value of 1. (FIG. 13B) KA values were expressed as the A/I ratio of huFcγRIIIa to huFcγRIIb/c for each of the four antibodies

FIGS. 14A-14D show ADCC mediated lysis of Raji-CD123 positive cells examined in a calcein release assay using normal PBMC as effector cells. Approximate numbers of CD123 molecules expressed on Raji-CD123 low and high expressors are 4,815 and 24,432 respectively. (FIG. 14A) ADCC-mediated lysis of Raji-CD123 low at E:T=25:1 (FIG. 14B) ADCC mediated lysis of Raji-CD123 low at E:T=50:1 (FIG. 14C) ADCC mediated lysis of Raji-CD123 high at E:T=25:1. (FIG. 14D) ADCC mediated lysis of Raji-CD123 high at E:T=50:1. Filled triangles represent hCSL360Fc3, circles hCSL360kif, filled circles CSL360, squares hCSL360, asterisk represents no antibody.

FIGS. 15A-15B show enhanced ADCC activity of CSL360 and its variants with TF-1 cells as target cells. ADCC activity of antibodies were examined using LDH assay. (FIG. 15A) Filled triangles represent hCSL360Fc3, filled squares hCSL360Fc2, empty circles hCSL360kif, filled circles CSL360 and asterisk represents no antibody. (FIG. 15B) Filled triangles represent 168-26Fc3, filled squares 168-26Fc2, filled circles represent 168-26 and asterisk represents no antibody

FIGS. 16A-16G show enhanced ADCC activity of CSL360 and its variants with primary human leukaemic cells as target cells, (FIG. 16A) RMH003 AML, (FIG. 16B) RMH011 AML, (FIG. 16C) RMH010 AML, (FIG. 16D) RMH008 AML, (FIG. 16E) WMH007 AML, (FIG. 16F) RMH009 B-ALL, (FIG. 16G) RMH007 B-ALL. ADCC activity was determined using LDH assay.

FIG. 17 shows in vivo sensitivity of mice with pre-engrafted ALL to control MAb (murine IgG2a), 7G3, 168-26 and 168-26Fc3 depicted as Kaplan-Meier curves for event-free survival (EFS) from the day of leukemic transplantation. An event is defined as 25% hCD45+ burden in peripheral blood. The number of animals in each group were 7, 6, 6 and 7 respectively. Leukemic growth delay (LGD) is defined as the number of days a treated group survived more than the control MAb group based on comparison of median EFS and were 2.9 (P=0.54), 6.4 (P=0.13) and 12.2 (P=0.044) days for 7G3, 168-26 and 168-26Fc3 respectively.

### DETAILED DESCRIPTION OF THE INVENTION

In one aspect, the present invention provides a method for inhibition of leukemic stem cells expressing IL-3Rα (CD123), which comprises contacting said cells with an antigen binding molecule comprising a Fc region or a modified Fc region having enhanced Fc effector function, wherein said antigen binding molecule binds selectively to IL-3Rα (CD123).

In this aspect, the invention also provides a method for the treatment of a hematologic cancer condition in a patient, which comprises administration to the patient of an effective amount of an antigen binding molecule comprising a Fc region or a modified Fc region having enhanced Fc effector function, wherein said antigen binding molecule binds selectively to IL-3Rα (CD123).

Preferably, the patient is a human.

The antigen binding molecule is preferably a monoclonal antibody or antibody fragment comprising a Fc region or a modified Fc region having enhanced Fc effector function.

Antibodies provide a link between the humoral and the cellular immune system with IgG being the most abundant serum immunoglobulin. While the Fab regions of the antibody recognize antigens, the Fc portion binds to Fcγ receptors (Fcγ Rs) that are differentially expressed by all immune accessory cells such as natural killer (NK) cells, neutrophils, mononuclear phagocytes or dendritic cells. Such binding crosslinks FcR on these cells and they become activated as a result. Activation of these cells has several consequences; for example, NK cells kill cancer cells and also release cytokines and chemokines that can inhibit cell proliferation and tumour-related angiogenesis, and increase tumour immunogenicity through increased cell surface expression of major histocompatibility antigens (MHC) antigens. Upon receptor crosslinking by a multivalent antigen/antibody complex, effector cell degranulation and transcriptional-activation of cytokine-encoding genes are triggered and is followed by cytolysis or phagocytosis of the target cell.

The effector functions mediated by the antibody Fc region can be divided into two categories: (1) effector functions that operate after the binding of antibody to an antigen (these functions involve, for example, the participation of the complement cascade or Fc receptor (FcR)-bearing cells); and (2) effector functions that operate independently of antigen binding (these functions confer, for example, persistence in the circulation and the ability to be transferred across cellular barriers by transcytosis). For example, binding of the C1 component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with an Fc receptor binding site on the antibody Fc region binding to a Fc receptor (FcR) on a cell. Binding of antibody to Fc receptors on cell surfaces triggers a number of important and diverse biological responses including engulfment and destruction of antibody-coated particles, clearance of immune complexes, lysis of antibody-coated target cells by killer cells (known as antibody-dependent cell-mediated cytotoxicity, or ADCC), release of inflammatory mediators, placental transfer and control of immunoglobulin production.

The present inventors have shown that the presence in the antigen binding molecule of a Fc region or a modified Fc region having enhanced Fc effector function is important for inhibition of leukemic stem cells expressing CD123, and hence in treatment of hematologic cancer conditions associated with leukemic stem cells.

The hematologic cancer conditions associated with leukemic stem cells (LSCs) which may be treated in accordance with the present invention include leukemias (such as acute myelogenous leukemia, chronic myelogenous leukemia, acute lymphoid leukemia, chronic lymphoid leukemia and myelodysplastic syndrome) and malignant lymphoproliferative conditions, including lymphomas (such as multiple myeloma, non-Hodgkin's lymphoma, Burkitt's lymphoma, and small cell- and large cell-follicular lymphoma).

As used herein the term “antigen binding molecule” refers to an intact immunoglobulin, including monoclonal antibodies, such as chimeric, humanized or human monoclonal antibodies, or to an antigen-binding and/or variable-domain-comprising fragment of an immunoglobulin that competes with the intact immunoglobulin for specific binding to the binding partner of the immunoglobulin, e.g. a host cell protein. Regardless of structure, the antigen-binding fragment binds with the same antigen that is recognized by the intact immunoglobulin. Antigen-binding fragments may be produced synthetically or by enzymatic or chemical cleavage of intact immunoglobulins or they may be genetically engineered by recombinant DNA techniques. The methods of production of antigen binding molecules and fragments thereof are well known in the art and are described, for example, in Antibodies: A Laboratory Manual, Edited by E. Harlow and D, Lane (1988), Cold Spring Harbor Laboratory, Cold Spring Harbor, N.Y., which is incorporated herein by reference.

The term “inhibition” as used herein, in reference to leukemic stem cells, includes any decrease in the functionality or activity of the LSCs (including growth or proliferation and survival activity), in particular any decrease or limitation in the ability of the LSCs to survive, proliferate and/or differentiate into progenitors of leukemia or other malignant hyperproliferative hematologic cancer cells.

The term “binds selectively”, as used herein, in reference to the interaction of a binding molecule, e.g. an antibody, and its binding partner, e.g. an antigen, means that the interaction is dependent upon the presence of a particular structure, e.g. an antigenic determinant or epitope, on the binding partner. In other words, the antibody preferentially binds or recognizes the binding partner even when the binding partner is present in a mixture of other molecules or organisms.

The term “effective amount” refers to an amount of the binding molecule as defined herein that is effective for treatment of a hematologic cancer condition.

The term “treatment” refers to therapeutic treatment as well as prophylactic or preventative measures to cure or halt or at least retard progress of the condition. Those in need of treatment include those already afflicted with a hematologic cancer condition as well as those in which such a condition is to be prevented. Subjects partially or totally recovered from the condition might also be in need of treatment. Prevention encompasses inhibiting or reducing the onset, development or progression of one or more of the symptoms associated with a hematologic cancer condition.

In the method of the present invention, administration to the patient of a chemotherapeutic agent may be combined with the administration of the antigen binding molecule, with the chemotherapeutic agent being administered either prior to, simultaneously with, or subsequent to, administration of the antigen binding molecule.

Preferably, the chemotherapeutic agent is a cytotoxic agent, for example a cytotoxic agent selected from the group consisting of:
(a) Mustard gas derivatives: Mechlorethamine, Cyclophosphamide, Chlorambucil, Melphalan, and Ifosfamide
(b) Ethylenimines: Thiotepa and Hexamethylmelamine
(c) Alkylsulfonates: Busulfan
(d) Hydrazines and triazines: Althretamine, Procarbazine, Dacarbazine and Temozolomide
(e) Nitrosureas: Carmustine, Lomustine and Streptozocin
(f) Metal salts: Carboplatin, Cisplatin, and Oxaliplatin
(g) Vinca alkaloids: Vincristine, Vinblastine and Vinorelbine
(h) Taxanes: Paclitaxel and Docetaxel
(i) Podophyllotoxins: Etoposide and Tenisopide.
(j) Camptothecan analogs: Irinotecan and Topotecan
(k) Anthracyclines: Doxorubicin, Daunorubicin, Epirubicin, Mitoxantrone and Idarubicin
(l) Chromomycins: Dactinomycin and Plicamycin
(m) Miscellaneous antitumor antibiotics: Mitomycin and Bleomycin
(n) Folic acid antagonists: Methotrexate
(o) Pyrimidine antagonists: 5-Fluorouracil, Foxuridine, Cytarabine, Capecitabine, and Gemcitabine
(p) Purine antagonists: 6-Mercaptopurine and 6-Thioguanine
(q) Adenosine deaminase inhibitors: Cladribine, Fludarabine, Nelarabine and Pentostatin
(r) Topoisomerase I inhibitors: Ironotecan and Topotecan
(s) Topoisomerase II inhibitors: Amsacrine, Etoposide, Etoposide phosphate and Teniposide
(t) Ribonucleotide reductase inhibitors: Hydroxyurea
(u) Adrenocortical steroid inhibitors: Mitotane
(v) Enzymes: Asparaginase and Pegaspargase
(w) Antimicrotubule agents: Estramustine
(x) Retinoids: Bexarotene, Isotretinoin and Tretinoin (ATRA).

Other examples of chemotherapeutic agents include, but are not limited to: acivicin; aclarubicin; acodazole hydrochloride; acronine; adozelesin; aldesleukin; altretamine; ambomycin; ametantrone acetate; aminoglutethimide; anastrozole; anthracyclin; anthramycin; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; brequinar sodium; bropirimine; cactinomycin; calusterone; caracemide; carbetimer; carrnustine; carubicin hydrochloride; carzelesin; cedefingol; cirolemycin; crisnatol mesylate; decitabine (Dacogen); demethylation agents; dexormaplatin; dezaguanine; dezaguanine mesylate; diaziquone; droloxifene; droloxifene citrate; dromostanolone propionate; duazomycin; edatrexate; eflornithine hydrochloride; EphA2 inhibitors; elsamitrucin; enloplatin; enpromate; epipropidine; erbulozole; esorubicin hydrochloride; etanidazole; etoprine; fadrozole hydrochloride; fazarabine; fenretinide; floxuridine; flurocitabine; fosquidone; fostriecin sodium; histone deacetylase inhibitors (HDAC-Is); ilmofosine; imatinib mesylate (Gleevec, Glivec); iproplatin; lanreotide acetate; lenalidomide (Revlimid); letrozole; leuprolide acetate; liarozole hydrochloride; lometrexol sodium; lomustine; losoxantrone hydrochloride; masoprocol; maytansine; megestrol acetate; melengestrol acetate; menogaril; metoprine; meturedepa; mitindomide; mitocarcin; mitocromin; mitogillin; mitomalcin; mitosper; mycophenolic acid; nocodazole; nogalamycin; ormaplatin; oxisuran; peliomycin; pentamustine; peplomycin sulfate; perfosfamide; pipobroman; piposulfan; piroxantrone hydrochloride; plomestane; porfimer sodium; porfiromycin; prednimustine; puromycin; puromycin hydrochloride; pyrazofurin; riboprine; rogletimide; safingol; saflngol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teroxirone; testolactone; thiamiprine; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vindesine; vindesine sulfate; vinepidine sulfate; vinglycinate sulfate; vinleurosine sulfate; vinrosidine sulfate; vinzolidine sulfate; vorozole; zeniplatin; zinostatin; zorubicin hydrochloride; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; anagrelide; anastrozole; andrographolide; angiogenesis inhibitors; antagonist D; antagonist G; antarelix; antiandrogen, prostatic carcinoma; antiestrogen; antineoplaston; antisense oligonucleotides; aphidicolin glycinate; apoptosis gene modulators; apoptosis regulators; apurinic acid; ara-CDP-D L-PTBA; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epristeride; estramustine analogue; estrogen agonists; estrogen antagonists; etanidazole; exemestane; fadrozole; fazarabine; fenretinide; filgrastim; finasteride; flavopiridol; flezelastine; fluasterone; fluorodaunorunicin hydrochloride; forfenimex; formestane; fostriecin; fotemustine; gadolinium texaphyrin; gallium nitrate; galocitabine; ganirelix; gelatinase inhibitors; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idoxifene; idramantone; ilmofosine; ilomastat; imidazoacridones; imiquimod; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leuprolide and, estrogen, and progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; matrilysin inhibitors; matrix metal loproteinase inhibitors; menogaril; merbarone; meterelin; metoclopramide; MIF inhibitor; mifepristone; miltefosine; mirimostim; mismatched double stranded RNA; mitoguazone; mitolactol; mitonafide; mitotoxin fibroblast growth factor-saporin; mofarotene; molgramostim; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; O6-benzylguanine; octreotide; okicenone; oligonucleotides; onapristone; oracin; oral cytokine inducer; ormaplatin; osaterone; oxaunomycin; paclitaxel; paclitaxel analogues; paclitaxel derivatives; palauamine; palmitoylrhizoxin; pamidronic acid; panaxytriol; panomifene; parabactin; pazelliptine; peldesine; pentosan polysulfate sodium; pentrozole; perflubron; perfosfamide; perillyl alcohol; phenazinomycin; phenylacetate; phosphatase inhibitors; picibanil; pilocaine hydrochloride; pirarubicin; piritrexim; placetin A; placetin B; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, sizofiran; sobuzoxane; sodium borocaptate; sodium phenylacetate; solverol; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; leucovorin; tamoxifen methiodide; tauromustine; tazarotene; tecogalan sodium; tegafur; tellurapyrylium; telomerase inhibitors; temoporfin; tetrachlorodecaoxide; tetrazomine; thaliblastine; thiocoraline; thrombopoietin; thrombopoietin mimetic; thymalfasin; thymopoietin receptor agonist; thymotrinan; tin ethyl etiopurpurin; tirapazamine; titanocene bichloride; topsentin; toremifene; totipotent stem cell factor; translation inhibitors; triacetyluridine; triciribine; trimetrexate; triptorelin; tropisetron; turosteride; tyrosine kinase inhibitors; tyrphostins; UBC inhibitors; ubenimex; urokinase receptor antagonists; vapreotide; variolin B; vector system, erythrocyte gene therapy; thalidomide; velaresol; veramine; verdins; verteporfin; vinxaltine; vorozole; zanoterone; zeniplatin; zilascorb; and zinostatin stimalamer.

In accordance with the present invention, the antigen binding molecule comprising a Fc region or a modified Fc region having enhanced Fc effector function is preferably administered to a patient by a parenteral route of administration. Parenteral administration includes any route of administration that is not through the alimentary canal (that is, not enteral), including administration by injection, infusion and the like. Administration by injection includes, by way of example, into a vein (intravenous), an artery (intraarterial), a muscle (intramuscular) and under the skin (subcutaneous). The antigen binding molecule may also be administered in a depot or slow release formulation, for example, subcutaneously, intradermally or intramuscularly, in a dosage which is sufficient to obtain the desired pharmacological effect.

In one embodiment of the invention, the antigen binding molecule comprises a modified Fc region, more particularly a Fc region which has been modified to provide enhanced effector functions, such as enhanced binding affinity to Fc receptors, antibody-dependent cellular cytotoxicity (ADCC) and complement-dependent cytotoxicity (CDC). For the IgG class of antibodies, these effector functions are governed by engagement of the Fc region with a family of receptors referred to as the Fcγ receptors (FcγRs) which are expressed on a variety of immune cells. Formation of the Fc/FcγR complex recruits these cells to sites of bound antigen, typically resulting in signaling and subsequent immune responses. Methods for optimizing the binding affinity of the FcγRs to the antibody Fc region in order to enhance the effector functions, in particular to alter the ADCC and/or CDC activity relative to the “parent” Fc region, are well known to persons skilled in the art. By way of example only, procedures for the optimization of the binding affinity of a Fc region are described by Niwa et al.³⁴, Lazar et al.³⁵, Shields et al.³⁶ and Desjarlais et al³⁷. These methods can include modification of the Fc region of the antibody to enhance its interaction with relevant Fc receptors and increase its potential to facilitate antibody-dependent cell-mediated cytotoxicity (ADCC) and antibody-dependent cell-mediated phagocytosis (ADCP)³⁴. Enhancements in ADCC activity have also been described following the modification of the oligosaccharide covalently attached to IgG1 antibodies at the conserved Asn²⁹⁷ in the Fc region^{35,36}. Other methods include the use of cell lines which inherently produce antibodies with enhanced Fc effector function (e.g. Duck embryonic derived stem cells for the production of viral vaccines, WO/2008/129058; Recombinant protein production in avian EBX® cells, WO/2008/142124). Methods for enhancing CDC activity can include isotype chimerism, in which portions of IgG3 subclass are introduced into corresponding regions of IgG1 subclass (e.g. Recombinant antibody composition, US2007148165).

In another aspect, the present invention provides the use of an antigen binding molecule comprising a Fc region or a modified Fc region having enhanced Fc effector function in, or in the manufacture of a medicament for, the inhibition of leukemic stem cells expressing IL-3Rα (CD123), wherein said antigen binding molecule binds selectively to IL-3Rα (CD123).

In this aspect, the invention also provides the use of an antigen binding molecule comprising a Fc region or a modified Fc region having enhanced Fc effector function in, or in the manufacture of a medicament for, the treatment of a hematologic cancer condition in a patient, wherein said antigen binding molecule binds selectively to IL-3Rα (CD123).

In yet another aspect, the invention provides an agent for inhibition of leukemic stem cells expressing IL-3Rα (CD123), which comprises an antigen binding molecule comprising a Fc region or a modified Fc region having enhanced Fc effector function, wherein said antigen binding molecule binds selectively to the IL-3Rα (CD123).

In this aspect, the invention also provides an agent for the treatment of a hematologic cancer condition in a patient, which comprises an antigen binding molecule comprising a Fc region or a modified Fc region having enhanced Fc effector function, wherein said antigen binding molecule binds selectively to IL-3Rα (CD123).

The agent of this aspect of the invention may be a pharmaceutical composition comprising the antigen binding molecule together with one or more pharmaceutically acceptable excipients and/or diluents.

Compositions suitable for parenteral administration conveniently comprise a sterile aqueous preparation of the active component which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example as a solution in polyethylene glycol and lactic acid. Among the acceptable vehicles and solvents that may be employed are water, Ringer's solution, suitable carbohydrates (e.g. sucrose, maltose, trehalose, glucose) and isotonic sodium chloride solution. In addition, sterile, fixed oils are conveniently employed as a solvent or suspending medium. For this purpose, any bland fixed oil may be employed including synthetic mono- or di-glycerides. In addition, fatty acids such as oleic acid find use in the preparation of injectables.

The formulation of such therapeutic compositions is well known to persons skilled in this field. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. The use of such media and agents for pharmaceutically active substances is well known in the art, and it is described, by way of example, in Remington's Pharmaceutical Sciences, 18th Edition, Mack Publishing Company, Pennsylvania, USA. Except insofar as any conventional media or agent is incompatible with the active ingredient, use thereof in the pharmaceutical compositions of the present invention is contemplated. Supplementary active ingredients can also be incorporated into the compositions.

Throughout this specification and the claims which follow, unless the context requires otherwise, the word “comprise”, and or variations such as “comprises” or “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of Endeavour to which this specification relates.

The present invention is further illustrated by the following non-limiting Examples:

### Example 1

This Example demonstrates the ability of MAb 7G3 to exploit the overt differences in CD123 expression and function between AML-LSCs and HSCs. MAb 7G3 inhibits the IL-3 signaling pathway and proliferation of primary AML cells. In addition, the homing and engraftment of AML blasts in the NOD/SCID xenograft model is profoundly reduced by MAb 7G3, and LSC function is inhibited.

### Methods

### AML Patient Samples, Normal Hematopoietic Cells, and Cell Lines

Apheresis product, bone marrow or peripheral blood samples were obtained from newly diagnosed and relapsed patients with AML. Patient samples were collected after informed consent according to institutional guidelines and studies were approved by the Royal Adelaide Hospital Human Ethics Committee, Melbourne Health Human Research Ethics Committee, Research Ethics Board of the University Health Network, and the South Eastern Sydney & Illawarra Area Health Service Human Research Ethics Committee. Diagnosis was made using cytomorphology, cytogenetics, leukocyte antigen expression and evaluated according to the French-American-British (FAB) classification. Mononuclear cells were enriched by Lymphoprep or Ficoll density gradient separation and frozen in liquid nitrogen. Human cord blood and BM cells were obtained from full-term deliveries or consenting patients receiving hip replacement surgery or commercially from Cambrex (US), respectively, and processed as previously described³⁸.

### Proliferation Assays

AML cell growth responses to IL-3 or GM-CSF were measured by [³H]-thymidine assay as previously described³⁹. Briefly, 2×10⁴ mononuclear cells per well in 96 well plates were stimulated with IL-3 (1 nM) or GM-CSF (0.1 nM) in the presence of 0.001-10 nM 7G3 or isotype-matched control BM4 (IgG2a) in 200 μl IMDM+10% Heat Inactivated Fetal Calf Serum (HI-FCS) (Hyclone, Utah) for 48 hours at 37° C., 5% CO₂ with 0.5 μCi of ³H-thymidine (MP Biomedicals, NSW, Australia) added for the last 6 hours of culture. Cells were deposited onto glass fiber paper using a Packard Filtermate cell harvester (Perkin Elmer, Victoria, Australia) and counted using a Top Count (Perkin Elmer). All cytokines and antibodies were obtained commercially (R&D Systems, Minneapolis, Minn.) or supplied by CSL Limited (Melbourne, Australia).

### Cytokine Signaling

Phosphorylation of signaling proteins was detected by immunoprecipitation and immunoblots. TF-1 cells and AML MNC cells were washed and rendered quiescent in IMDM medium with 0.5% HI-FCS (Hyclone, Utah) or with 0.5% human albumin (CSL, Melbourne, Australia) in the absence of growth factors for 18 hours. One hundred million cells were incubated with IgG2a (100 nM), 9F5, 6H6 (non-blocking anti-CD123 antibodies), or 7G3 (0.0001-100 nM) for 30 min on ice, and then stimulated with 50 ng/mL IL-3 for 15 min at 37° C. Cells were lysed in NP-40 lysis buffer⁴⁰ and human β_{c} (CD131) was immunoprecipitated using 1C1 and 8E4 antibodies conjugated to Sepharose beads. Immunoprecipitates were subjected to SDS-PAGE and immunoblotting as previously described⁴¹. Antibodies used to probe the immunoblots were: 4G10, antiphosphotyrosine MAbs (Upstate Biotech, Lake Placid, N.Y.); anti-phospho-Akt Ser473 (Cell Signaling, Beverly, Mass.); and anti-phosphorylated signal transducer and activator of transcription 5 (STAT-5) MAb (Zymed, San Francisco, Calif.). All antibodies were used according to manufacturer's instructions. Signals were developed using enhanced chemiluminescence (ECL; Amersham Pharmacia or West Dura from Pierce).

STAT-5 activation was also detected by intracellular FACS on leukemic cell lines M07e and TF1, and primary AML cells. Cells were incubated in MEDM plus 10% FCS and 10 ng/mL of huIL-3 (CSL, Melbourne, Australia) for 60 minutes, and fixed with BD Cytofix™ Buffer (Becton-Dickinson) followed by methanol permeabilization. Cells were then stained with anti-phosphoSTAT-5 (Becton-Dickinson) and analyzed using a FACSCalibur (Becton-Dickinson) instrument.

### Ex Vivo Antibody Treatment

Thawed AML or normal hematopoietic cells were incubated with control IgG2a or 7G3 (10 μg/mL) for 2 hours in X-VIVO 10 (Cambrex BioScience) supplemented with 15-20% BIT (StemCell Technologies, Vancouver, BC Canada)) at 37° C. before intravenous transplantation into sub-lethally irradiated NOD/SCID mice for repopulating assays (see below). Engraftment was measured at 4-10 weeks at 2 different time points.

### In Vivo Antibody Treatment of AML

For in vivo testing, control IgG2a or 7G3 (300-500 μg per injection) were injected intraperitoneally (i.p.) into mice 3 times a week with schedules described in the legends to each figure. To investigate possible synergistic effects of 7G3 with cytarabine (Ara-C), 35 days post-transplantation, 500 μg of antibodies were injected once a day for 3 consecutive days followed by i.p. injection of Ara-C at 40 mg/kg/d for 5 consecutive days. Antibody treatments resumed at 500 μg per injection 3 times a week for another 4 weeks following which engraftment was measured 3 days after the last injection of antibody.

Xenotransplantion of Human Cells into NOD/SCID Mice

Animal studies were performed under the institutional guidelines approved by the University Health Network/Princess Margaret Hospital Animal Care Committee or the Animal Care and Ethics Committee of the University of New South Wales. Transplantation of human cells into NOD/SCID mice was performed as previously described³⁸. Briefly, all mice received sublethal irradiation (250-350 cGy) 24 hours before intravenous (i.v.) or intrafemoral transplantation with 5-10 million human cells per mouse. Anti-CD122 antibody was purified from the hybridoma cell line TM-β1 (generously provided by Prof. T. Tanaka, Hyogo University of Health Sciences)⁴² and 200 μg was injected i.p. into mice immediately after irradiation for natural killer cell depletion as previously described⁴³. Similarly, 8 million normal bone marrow cells, or 1 million sorted CD34⁺ normal bone marrow cells, or 3×10⁵ lineage depleted CD34⁺ normal cord blood cells were transplanted i.v. per mouse. Engraftment levels of human AML and normal hematopoietic cells in the murine bone marrow, peripheral blood, liver and spleen were evaluated based on the percentage of hCD45⁺ cells by flow cytometry. To measure 7G3 effects on LSC activity, secondary transplantations were also performed by i.v. transplantation of identical numbers of human cells (9 million cells/mouse) isolated from the bone marrow of previously engrafted mice in the IgG2a or 7G3 treatment groups.

### Homing Assay

Identical numbers of human cells from primary patient samples or harvested from engrafted mice were injected i.v. into sublethally irradiated NOD/SCID mice. Sixteen-twenty-four hours after injection, mononucleated cells from bone marrow, spleen, and peripheral blood of the recipient mice were analyzed by flow cytometry for human cells using 5×10⁴-1×10⁵ collected events. Homing efficiency of human cells into the mouse tissues was determined by measuring the % of the injected cells found in specific organs, calculated by the formula: % of huCD45⁺ cells assessed in the tissue×total number of cells in the specific tissue/total number of injected human cells×100⁴⁴⁻⁴⁶.

### Cell Staining and Flow Cytometry

Cells from the bone marrow, spleen, liver and peripheral blood of treated mice were stained with fluorescein isothiocyanate (FITC)-conjugated antimurine and phycoerythrin-cyanin 5 (PC5, Beckman-Coulter) or allophycocyanin (APC, BioLegend and Becton-Dickinson) conjugated anti-human antibodies, as previously described². CD123 expression was measured with phycoerythrin (PE) conjugated anti-human CD123 antibody (clone 9F5). 7G3 binding on human cells recovered from 7G3 treated mice was measured by staining duplicate samples with 9F5-PE or 7G3-PE, since the two clones bind to completely separate epitopes and produce similar levels of fluorescence on untreated primary cells (data not shown). The level of 7G3 binding was calculated by the formula: [(RFI of 9F5-PE detected CD123)−(RFI of 7G3-PE detected CD123)]÷(RFI of 9F5-PE detected CD123)×100. Immunophenotype and stem cell population were identified using a range of anti-human antibodies: anti-CD15-FITC, anti-CD14 conjugated to PE, anti-CD19-PE, anti-CD33-PE, anti-CD34-FITC or anti-CD34-PC5, and anti-CD38-PE or PE-Cyanine 7 (all antibodies from Becton-Dickinson unless otherwise stated). Isotype control antibodies were used to exclude 99.9% of negative cells, and cells were analyzed using FACScan or FACS Calibur flow cytometers (Becton-Dickinson).

### Statistical Analysis

Data are presented as the mean±s.e.m. The significance of the differences between groups was determined by using Student's t-test.

### Results

### Monoclonal Antibody 7G3 Blocks IL-3-Mediated Signaling in IL-3-Dependent Cell Lines and Primary AML Cells.

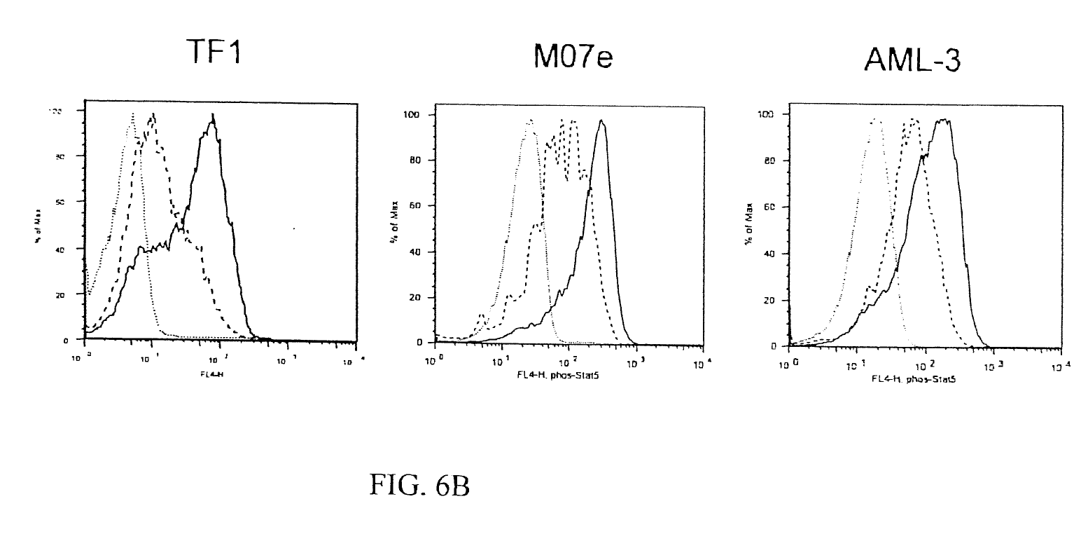

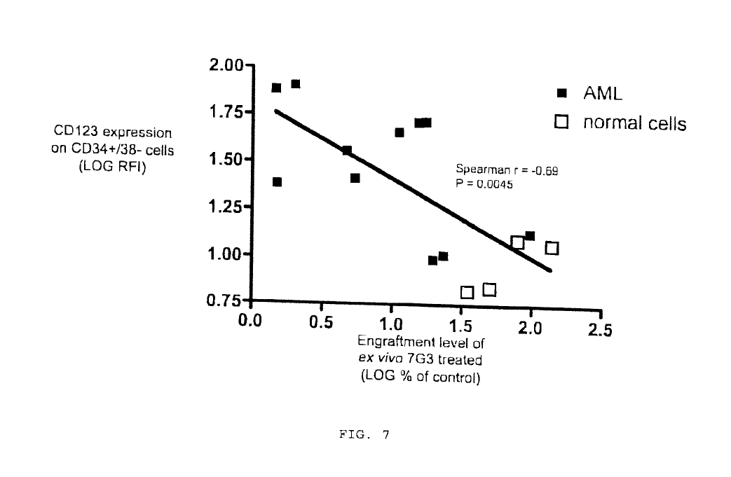

The monoclonal antibody 7G3, raised against the IL-3Receptor α subunit (IL-3Rα, CD123), has previously been shown to inhibit IL-3 binding to CD123 as well as IL-3-mediated effects in vitro, including proliferation of a leukemic cell line (TF-1), histamine release from human basophils, and endothelial cell activation³³. Consistent with these findings it has now been found that MAb 7G3 inhibited intracellular signaling in TF-1 cells and primary human AML cells. Stimulation of growth factor-deprived TF-1 cells with IL-3 (1 nM) resulted in tyrosine phosphorylation of the receptor β subunit (CD131), and activation of the STAT-5 and Akt downstream signaling molecules that play a role in cell proliferation and survival (FIG. 6a). CD131 tyrosine phosphorylation, and STAT-5 and Akt activation, were inhibited by incubation of cells with 7G3 at 1 nM, further reduced at 10 nM, and completely blocked at 100 nM concentration consistent with a reported Kd of 900 pM for 7G3³³. Two poorly neutralizing antibodies to CD123 that do not block IL-3 binding, 9F5 and 6H6, were ineffective at inhibiting IL-3-mediated signaling (FIG. 6a). The inhibition of IL-3-stimulated phosphorylation of STAT-5 by 7G3 in IL-3-dependent leukemic cell lines TF-1 and MO7e was also demonstrated by a flow cytometric assay (FIG. 6b). Importantly, MAb 7G3 selectively inhibited the IL-3-dependent phosphorylation of tyrosine 577 of CD131, a signal involved in promoting cell survival⁴⁰, in primary AML cells in a concentration-dependent manner (FIG. 6a). Similarly, 7G3 also reduced IL-3-stimulated STAT-5 phosphorylation in primary AML cells, as measured by flow cytometry (FIG. 6b). This selective inhibition of IL-3 signaling by MAb 7G3 is consistent with its ability to block IL-3 binding and raised the important question of whether the leukemic stem cell, previously reported not to express CD131 (β chain)²⁵, could be signaling exclusively through CD123 (α chain).

CD123 (IL-3Receptor α Chain) is Co-Expressed with CD131 (Receptor β Chain) on AML Leukemic Stem Cells

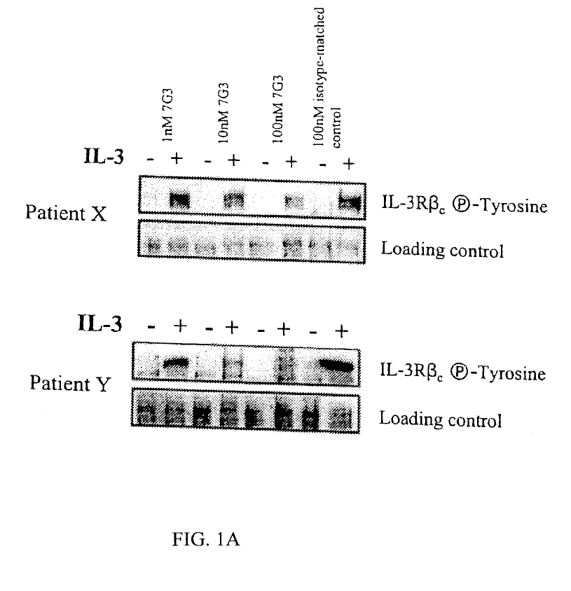

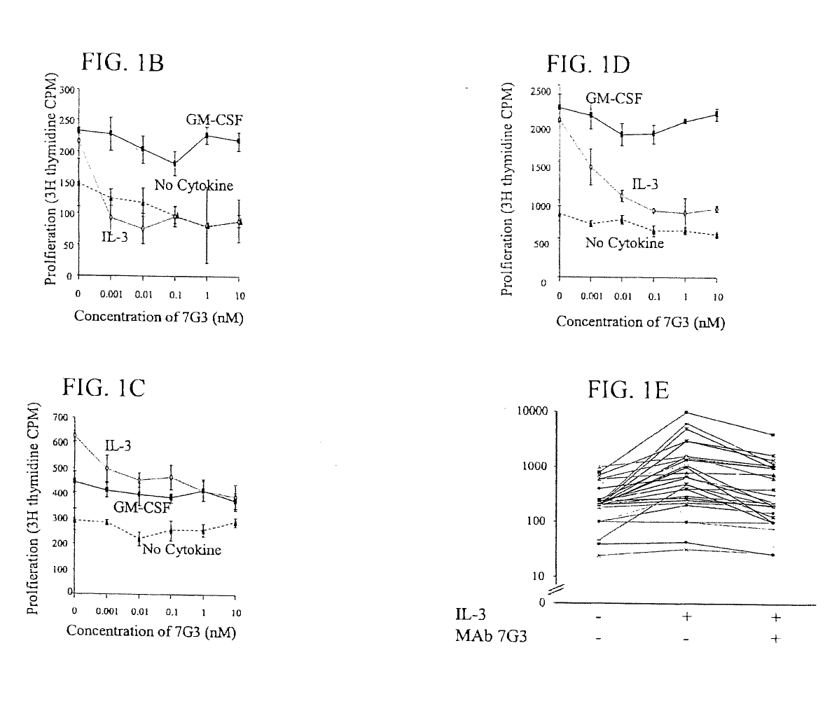

Overexpression of CD123 on CD34⁺/CD38⁻ cells from AML patients has been widely reported¹⁷⁻²¹ and has been proposed as a marker of leukemic CD34⁺/CD38⁻ stem cells (LSCs) in some studies^{24,25}. In the current study, CD123 expression on multiple AML samples was measured independently at 2 different laboratories. CD123 expression on AML CD34⁺/CD38⁻ cells (RFI 67.7±24.2, n=9) was significantly higher than that on normal hematopoietic CD34⁺/CD38⁻ cells (RFI 17.1±8.6, n=4, P=0.21, (data summarized in Table 1 below), consistent with other reports^{17-21,24,25}. This overexpression appeared to be selective, in that the GM-CSF receptor α chain (CD116) was not expressed in the equivalent population in AML samples as measured by flow cytometry. Instead, the GM-CSF receptor α chain was abundantly expressed on CD34⁻ blast cells (data not shown). Furthermore, flow cytometry and PCR analyses demonstrated that CD34⁺ cells that express CD123 also express CD131 (data not shown) suggesting that signal transduction occurs through the classical heterdimeric IL-3Receptor and not through CD123 alone, which is also supported by the CD131 phosphorylation data (FIG. 1a). Moreover, the difference in CD123 expression levels between normal and malignant CD34⁺/CD38⁻ progenitor cells provides the basis for 7G3 to selectively target LSC but not normal hematopoietic stem cells.

### 7G3 Inhibits Spontaneous and IL-3-Induced Proliferation of Primary AML Samples In Vitro

The ability of 7G3 to inhibit IL-3-induced proliferation was investigated using 38 primary AML patient samples. Representative plots for 3 primary samples are shown in FIG. 1b-d. 7G3 inhibited IL-3-induced proliferation in 32/35 samples (FIG. 1e), but not GM-CSF-stimulated growth (FIG. 1b-d). In the absence of exogenously added growth factors, 7G3 also inhibited the growth of cells from some AML samples. In 9 of the primary samples tested, the presence of 7G3 and IL-3 reduced the proliferation to ˜60% of endogenous levels with a range of 50-75% (FIG. 1e), suggesting an autocrine pathway. The poorly blocking 6H6 antibody did not inhibit IL-3-induced proliferation (data not shown). The Kd of the 7G3 antibody (approx 900 pM)³³ fitted well with the concentrations required to inhibit proliferation (FIG. 1 b-d). Overall, 7G3 was effective in inhibiting IL-3-mediated growth in the majority of primary AML samples, as well as spontaneous growth (no IL-3 added), suggesting that either some AML cells constitutively produce IL-3 or that 7G3 triggers a negative signal in these cells.

Pretreatment with 7G3 Inhibits AML but not Normal Hematopoietic Cell Engraftment in NOD/SCID Mice

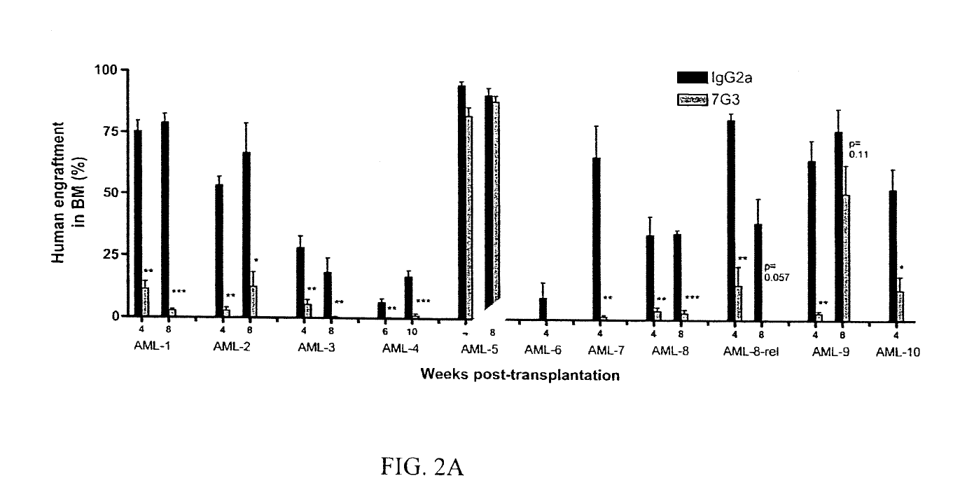

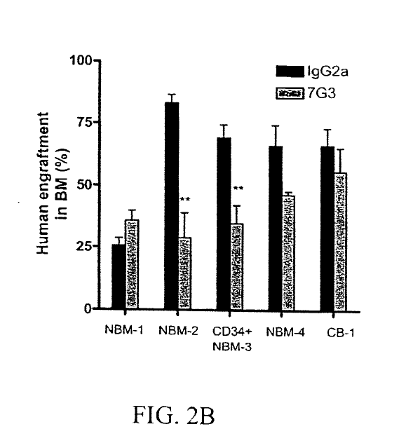

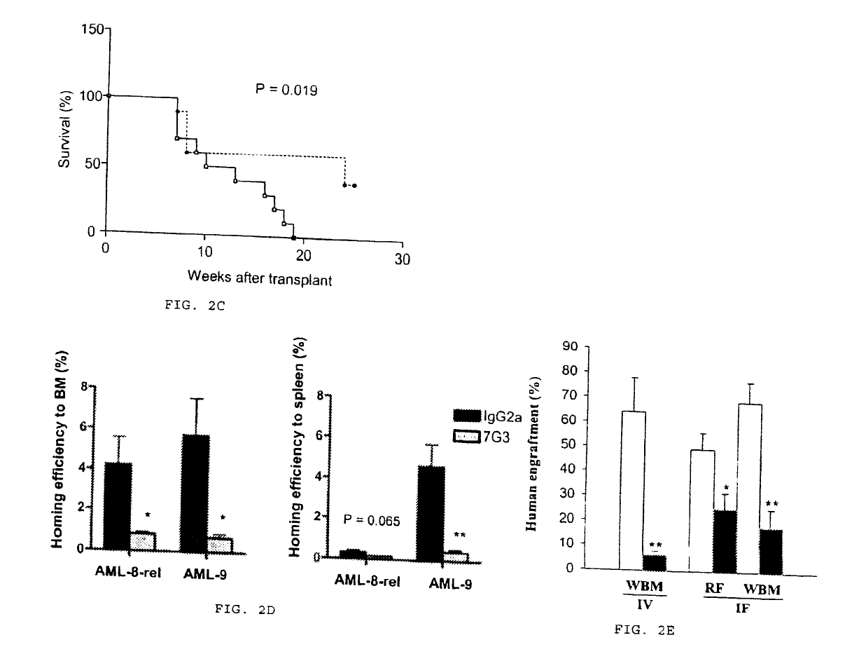

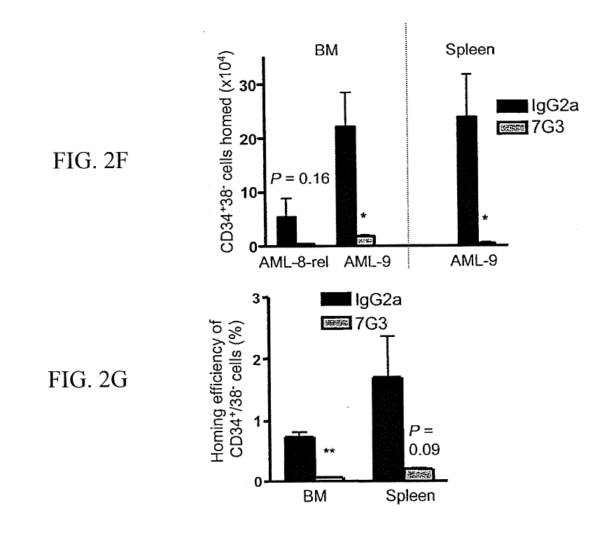

To assess the effects of 7G3 on the ability of normal and malignant cells to repopulate in immune-deficient mice, primary AML and normal bone marrow (NBM) or umbilical cord blood (CB) cells were incubated ex vivo with 7G3 or irrelevant IgG2a (10 μg/mL, 2 h) and transplanted into sub-lethally irradiated NOD/SCID mice. Ex vivo 7G3 incubation markedly reduced the engraftment of 9/10 primary AML samples whose controls showed evidence of bone marrow engraftment at 4-8 weeks post-inoculation (mean 89.7±1.9% reduction relative to controls, P=0.013, FIG. 2a and Table 1). This reduction in engraftment was sustained in 6/7 of the samples when assessed between 8 and 12 weeks following inoculation. In contrast, at 4-11 weeks post-inoculation, 7G3 had no significant inhibitory effects on the engraftment of 3/5 normal samples, and while small effects against two NBMs reached statistical significance, the inhibition was much less marked compared to AML cells (FIG. 2b and Table 1). Ex vivo 7G3 treatment reduced normal hematopoietic cell engraftment by an average of 23.5±8.9% (P=0.078) relative to IgG2a controls. Multi-lineage engraftment for 3 of the NBMs was measured by monitoring CD33, CD19, and CD3 expression, and no significant differences were found between the IgG2a and 7G3 treatment groups (data not shown).

Ex vivo 7G3 treatment inhibited to a similar extent the engraftment of AML-8 harvested at both diagnosis and relapse, indicating that both diagnosis and relapse samples may have comparable sensitivity to 7G3 treatment. AML-5 was the only AML sample in which engraftment was not reduced by ex vivo 7G3 treatment, which could be attributed to this sample exhibiting a high proportion of LSC (CD34⁺/CD38⁻) and the lowest CD123 expression of all the AML samples evaluated (Table 1). Overall, these results demonstrate the reduced sensitivity of normal hematopoietic stem cells to 7G3 treatment in comparison with AML LSC.

The reduction in AML engraftment caused by ex vivo 7G3 treatment was also associated with improved survival. Mice transplanted with IgG2a or 7G3 treated AML-9 cells exhibited median survival of 11.5 and 24 weeks, respectively (P=0.0188, n=10 for each group, FIG. 2c), with 40% of the 7G3 group surviving beyond the end of the experiment (25 weeks), in contrast with the control group in which no mice survived beyond 20 weeks.

The inhibitory effect of ex vivo 7G3 treatment on engraftment of AML or normal hematopoietic cells was inversely associated with the intensity of CD123 expression on the CD34⁺/CD38⁻ population, with a significant relationship (FIG. 7; R=−0.68, P=0.0051). A binary pattern was apparent, demonstrating that for those AML samples where engraftment was severely inhibited by 7G3 the CD123 expression was generally high. Conversely, the single AML sample (AML-5), along with the normal hematopoietic samples, for which engraftment was not as markedly affected by 7G3, generally expressed lower levels of CD123.

### 7G3 Inhibits AML Homing Capacity in NOD/SCID Mice

To determine the effects of 7G3 on the ability of intravenously-inoculated AML cells to home to the bone marrow and spleen, ex vivo-treated AML-8-rel and AML-9 cells were transplanted and mice were euthanased and examined 24 h later. 7G3 significantly diminished homing to the bone marrow to between 46-93% compared with isotype-treated controls (P<0.05), while homing to the spleen was reduced to 35 to 90% of control but the difference was not statistically significant (P>0.05) (FIG. 2d). The leukemic cells that resided in the bone marrow and spleen at 24 hours following inoculation were principally CD34⁺ primitive cells, and while 7G3 reduced the number of cells in the bone marrow, it did not alter the cell surface phenotype of the residing cells (data not shown).

To further characterize the effects of 7G3 on AML homing to the bone marrow, AML-8-rel cells were exposed to 7G3 or isotype control antibodies, and subsequently transplanted via the tail-vein (IV) or directly into the right femur (RF), and the animals euthanased 5 weeks thereafter. FIG. 2e shows that intra-femoral inoculation attenuated the inhibitory effects of 7G3 on engraftment compared with IV inoculated, although 7G3 remained effective at significantly reducing engraftment in both the injected femur and the non-injected femur. In order to more directly demonstrate 7G3 inhibition of AML-LSCs, we investigated the impact of 7G3 treatment on CD34⁺CD38⁻ cells since AML-LSCs (as defined by their ability to recapitulate the human disease in NOD/SCID mice) are significantly enriched in this fraction^{2,3}. The number of CD34⁺CD38⁻ cells from AML-8-rel and AML-9 homing to the BM was reduced by ex vivo 7G3 treatment to 8.4±0.018% and 12.0±4.3% of control, respectively (P=0.16 and 0.013, FIG. 2f). Similarly, the number of AML-9 CD34⁺CD38⁻ cells homing to the spleen was reduced to 3.8±1.5% of control (P=0.019). To further confirm this finding, the homing experiment was repeated with CD34⁺CD38⁻ cells sorted from AML-9 and then treated ex vivo with either IgG2a or 7G3 before injecting into NOD/SCID mice. The homing efficiency of human cells in the 7G3 treated group was reduced to 7.8±1.7% of IgG2a controls in the BM (P=0.0019) and 11.2±0.84% in the spleen (P=0.09) (FIG. 2g). Therefore, CD123 appears to play an important role in the homing of AML NOD/SCID leukemia-initiating cells (SL-ICs) to their supportive microenvironment, as well as establishment and dissemination of the disease in NOD/SCID mice.

### Early Administration of 7G3 Reduces AML Engraftment in NOD/SCID Mice

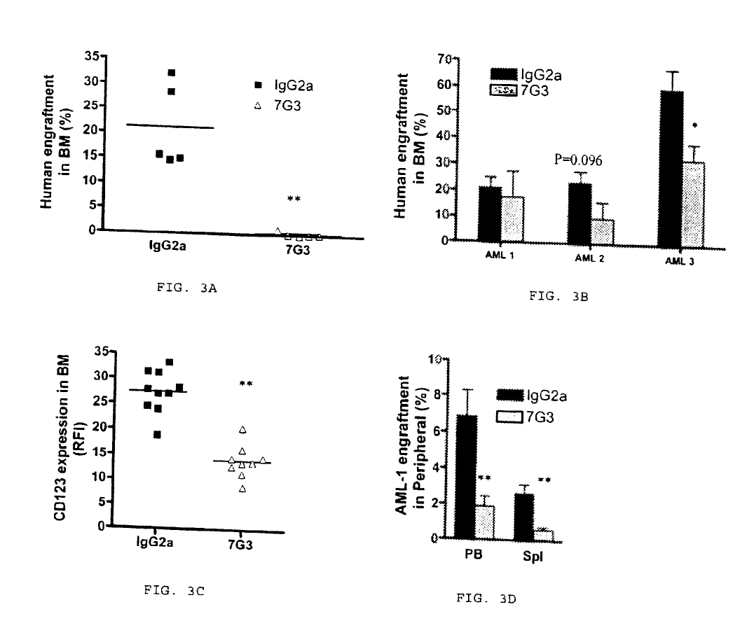

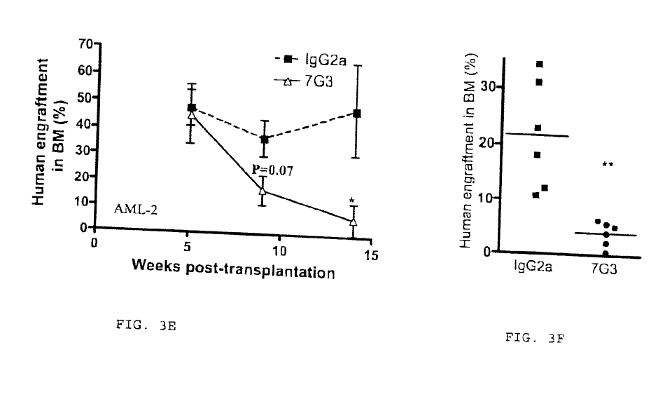

To determine whether 7G3 treatment of NOD/SCID mice affected AML cell engraftment, mice were administered a single intraperitoneal injection of 7G3 or isotype control antibodies (300 μg) followed by IV transplantation of AML-1 cells 6 hours later. 7G3 treatment almost completely ablated engraftment in the bone marrow, to 1.3±0.9% of control at 5 weeks post-transplantation (P=0.0006, n=5, FIG. 3a).

The efficacy of 7G3 in controlling the progression of AML in NOD/SCID mice was also examined by initiating treatments either 24 h or 4 days post-transplantation, presumably allowing the SL-IC to home to the bone marrow microenvironment before commencement of treatments⁴⁴⁻⁴⁶. When treatment was initiated 24 hours post-transplantation, engraftment was reduced in 2/3 AML samples. With this treatment regimen of 4 doses administered every other day, engraftment of AML-2 and -3 was reduced to 41.1±27.1% (P=0.096) and 39.6±10.0% (P=0.026) of controls, respectively, while engraftment of AML-1 was not affected (FIG. 3b).

Despite the relatively modest effects of 7G3 in both post-transplantation treatment regimens, 7G3 coating on AML cells harvested from the mouse bone marrow was clearly evident (data not shown). Moreover, 7G3 treatment decreased CD123 expression on AML-1 cells in any treatment regimen tested. For illustration, 7G3 treatment commencing 4 days post-transplantation decreased CD123 expression of AML-1 harvested from the BM to 51.3±4.0% of control (FIG. 3c, P<0.0001), as assessed using the 9F5 antibody. In the same experiment, 7G3 also reduced the dissemination of AML-1 to mouse peripheral blood and spleen to 27.8±7.5% (P=0.0029) and 23.5±5.3% (P=0.0009) of control, respectively (FIG. 3d).

### 7G3 can Reduce the Burden of Established AML Disease in NOD/SCID Mice

While the primary aim of this study was to test the effect of targeting CD123 on AML stem cells, the ability of 7G3 to exhibit any single agent therapeutic activity on established leukemic disease, above and beyond its effects on leukemic stem cell engraftment was evaluated by initiating continuous 7G3 or control IgG2a treatments 28 days post-transplantation in an established disease model, and continuing treatment until the time of sacrifice. There was variation in response to 7G3 treatment in this model between patient samples likely reflective of the heterogeneity of AML seen clinically. A significant reduction in the BM burden of AML was seen in 2 of 5 samples (shown in FIGS. 3e and f). AML-2 responded to 7G3 with a significant reduction in BM engraftment at 9 and 14 weeks post-transplantation (FIG. 3e), while treatment of mice with only 4 doses of 7G3 over 8 days significantly reduced the engraftment of AML-1 to 18.9±4.1% (P=0.001, FIG. 3f) of IgG2a control. Moreover, while a number of AML samples did not have a significant reduction in leukemic burden in the BM with initiation of 7G3 treatment at either 4 or 28 days post transplantation, it was generally observed that the leukemic burden in the peripheral hematopoietic organs (spleen, peripheral blood, and liver) was lower in the 7G3 treated group (FIG. 3d and data not shown). Together, these data suggest that 7G3 is biologically active in vivo and can repress the growth of AML in the NOD/SCID model when used as a single agent.

### 7G3 Targets SL-IC Self Renewal Capability

The serial transplantation experiments address an important question for all cancer stem cell (CSC)-directed therapies and provide evidence that the CSC is actually being targeted in vivo. In the case of AML, it is known that when AML-LSCs repopulate primary NOD/SCID mice they must self-renew³; self-renewal is a key property of all stem cells and is best assessed by secondary transplantation.

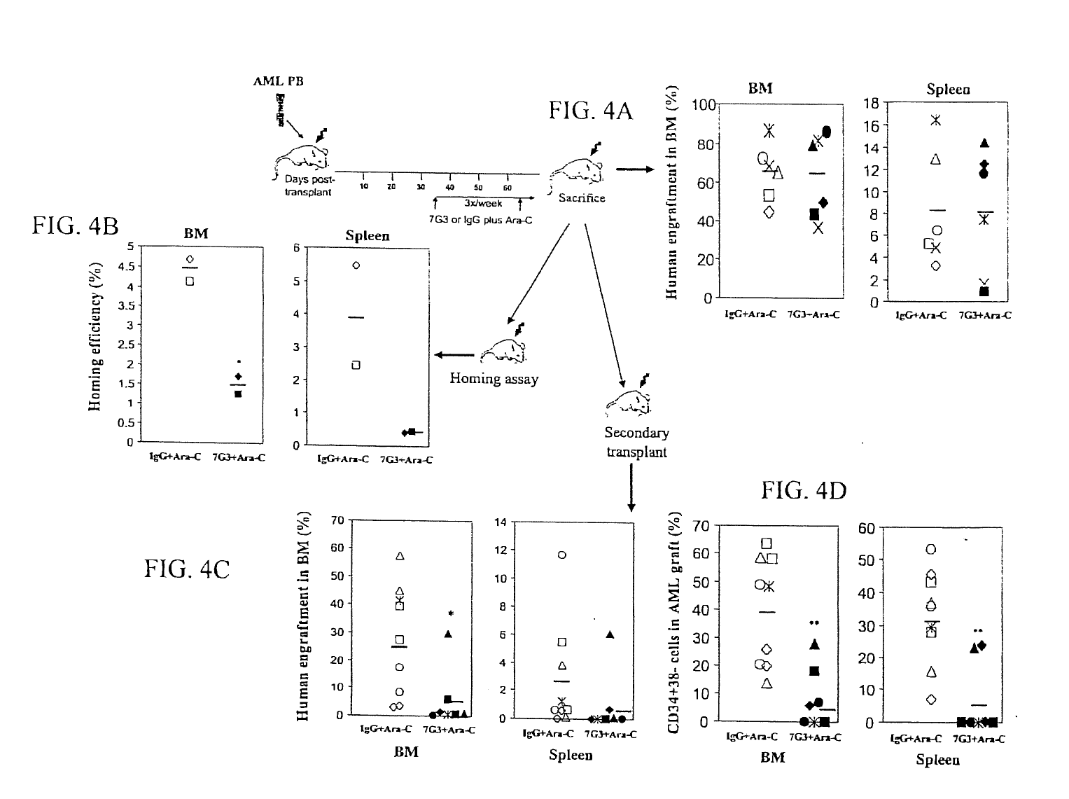

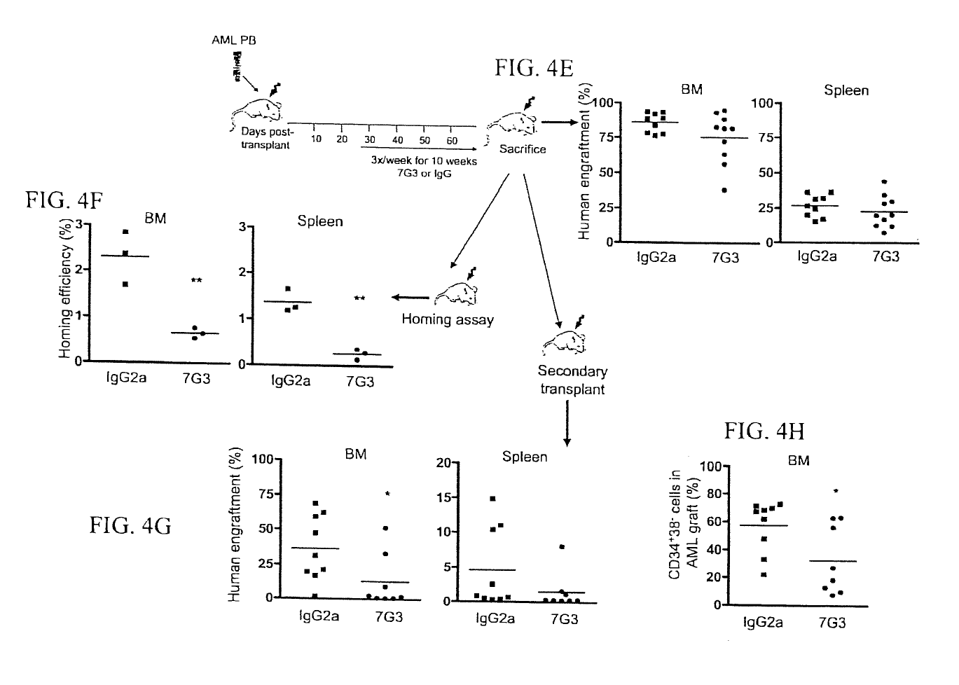

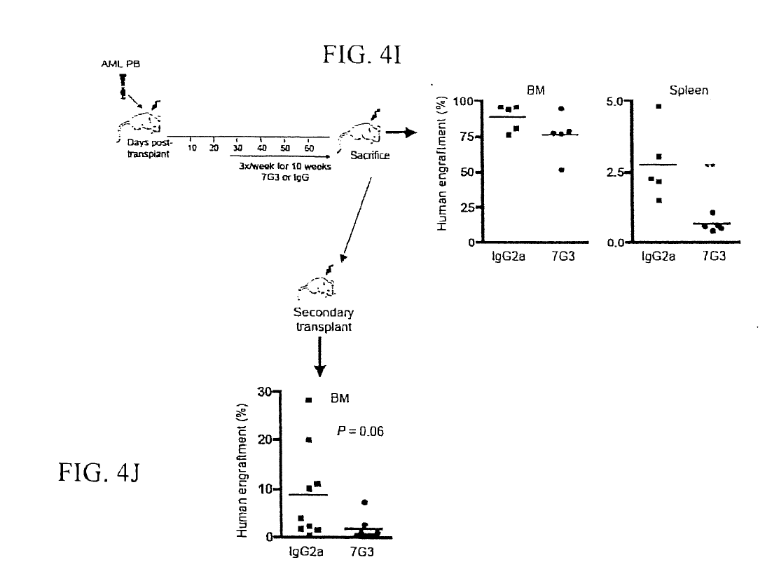

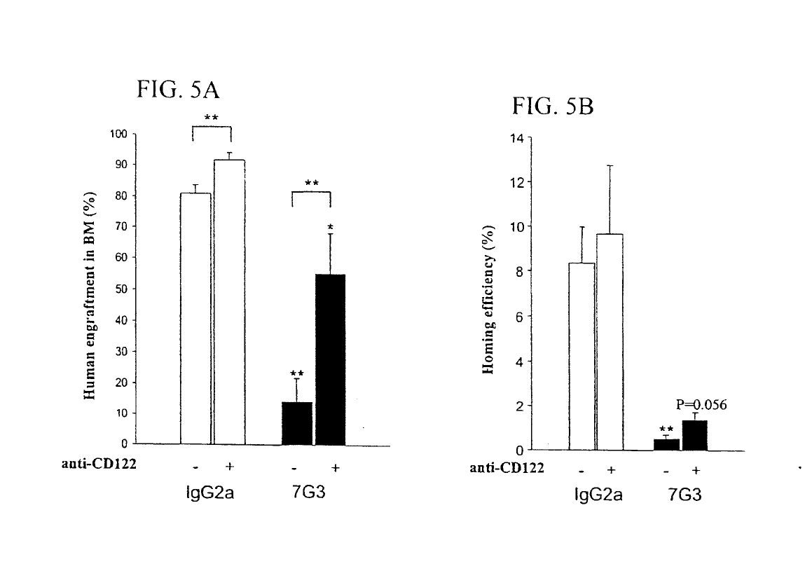

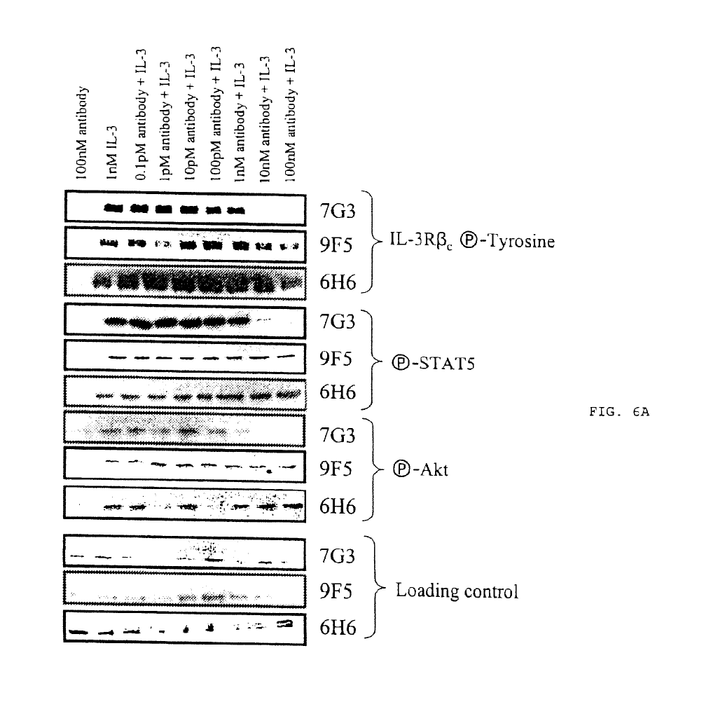

To examine whether 7G3 can also be used to target the LSC with self-renewal ability as an adjuvant to conventional therapy, which targets the more rapidly proliferating AML blasts, 7G3 or IgG2a were combined with cytarabine (Ara-C) and their effect on SL-IC and leukemic burden determined. At 35 days post transplantation with AML-10 cells, mice were treated with 7G3 or IgG2a control (500 μg/d) each day for 3 days followed by Ara-C (40 mg/kg/d) for 5 consecutive days. Following the Ara-C treatments, 7G3 was administered for another 4 weeks. Leukemic engraftment in the bone marrow and spleen of the mice treated with 7G3 and Ara-C was not decreased compared to mice treated with IgG2a and Ara-C (FIG. 4 Part Ia). However, when cells were harvested from the bone marrows of treated mice and equal numbers of human cells transplanted into secondary recipient mice, the homing of cells harvested from 7G3/Ara-C-treated donor mice to the bone marrow and spleen was inhibited to 33.6±5.0% (P=0.014) and 10.9±4.6% (P=0.15) of IgG2A/Ara-C-treated controls, respectively (FIG. 4 Part Ib). Moreover, repopulation of the bone marrow and spleen of secondary recipient mice was also reduced by 7G3/Ara-C to 21.0±15.2% (P=0.024) and 35.8±31.8% (P=0.31) of IgG2a/Ara-C-treated controls, respectively (FIG. 4 Part Ic). While the proportion of CD34⁺/CD38⁻ LSCs appearing in the bone marrow of donor mice was not decreased by 7G3/Ara-C relative to IgG2a/Ara-C treatment (data not shown), FIG. 4 Part Id shows a significant decrease in this cell population in the bone marrow and spleen of secondary recipient mice from 7G3/Ara-C donors compared with donors treated with IgG2a/Ara-C. These data demonstrate that in vivo 7G3 administration specifically targets AML-LSC in NOD/SCID mice, resulting in decreased homing and engraftment in secondary recipient mice.

To establish whether 7G3 can act as a single agent, serial transplantation was performed following in vivo 7G3 treatment in the absence of Ara-C. As shown in FIG. 4 Part II A, while weeks of 7G3 treatment did not overtly decrease the engraftment of AML-10 in the BM or spleen of primary engrafted mice, the AML cells harvested from 7G3-treated mice had significantly impaired homing ability to the BM (28.2±2.9%, P=0.0083) and spleen (18.3±4.8%, P=0.0021) of secondary recipient mice compared with IgG2a-treated controls (FIG. 4 Part II B). The repopulation ability was also significantly impaired: while 8 of 9 secondary recipient mice transplanted with untreated control cells were engrafted, only 3 of 8 mice inoculated with cells from 7G3-treated mice showed evidence of engraftment (FIG. 4 Part II C). The mean engraftment level in the 7G3 treated mice was significantly reduced compared with IgG2a treated controls (BM, 34.6±18.6%, P=0.039; spleen, 33.7±20.4%, P=0.19) (FIG. 4 Part II C). This patient sample had a high level of CD34⁺CD38⁻ primitive cells that was not decreased in the 7G3-treated primary mice. However, there was a significant decrease of this primitive cell population in the BM of secondary recipient mice transplanted from 7G3-treated donors compared with donors treated with IgG2a (56.6±15.0% of control, P=0.031) (FIG. 4 Part II D). Similar results were obtained in an independent experiment with AML-9 cells, showing that 7G3 caused a reduction in the mean level of engraftment to 19.3%±9.8% of control (FIG. 4 Part III).

Collectively, combining data from all 3 independent experiments depicted in FIG. 4, only 1 of 27 (3.7%) secondary mice was not engrafted by the cells harvested from IgG2a or IgG2a plus Ara-C treated control mice. By contrast, 11 of 23 (48%) secondary mice could not be engrafted by the cells harvested from 7G3 or 7G3 plus Ara-C treated mice. These results demonstrate that in vivo 7G3 administration specifically targets AML-LSCs in NOD/SCID mice, resulting in decreased homing and engraftment in secondary recipients.

### CD122⁺ NK Cells Contribute to 7G3-Mediated Inhibition of AML Repopulation in NOD/SCID Mice

NK cells, macrophages, neutrophils and dendritic cells are among the effector cells in the immune system that facilitate Fc-dependent, antibody-dependent cellular cytotoxicity (ADCC). Their contribution to the ability of 7G3 to inhibit engraftment of AML was assessed by injecting a monoclonal antibody against murine IL-2R β-chain (IL-2Rβ) also known as CD122 to irradiated NOD/SCID mice before leukemic cell transplantation of ex vivo 7G3-treated AML cells. IL-2Rβ is widely expressed on NK cells, T cells, and macrophages and blocking IL-2Rβ by mAb can improve the engraftment of human hematopoietic cells in the NOD/SCID xenotransplant system.

At 4 weeks post-transplantation, leukemic engraftment in the NK cell depleted mice transplanted with AML-8-rel cells treated ex vivo with IgG2a control was increased to 113.3±2.8% (P=0.023) of non-depleted mice (FIG. 5a), suggesting that CD122⁺ NK cells moderately decrease AML engraftment in NOD/SCID mice. Depletion of CD122⁺ cells also partially, but significantly, attenuated the ability of 7G3 to reduce engraftment of AML cells, suggesting that CD122 positive cells mediate, in part, the 7G3 inhibitory effect (FIG. 5a). In contrast to the effects on NOD/SCID repopulation, 7G3 still strongly inhibited the homing of leukemic cells by more than 85% of IgG control in the anti-CD122 treated mice (FIG. 5b). These results indicate that the ability of 7G3 to inhibit engraftment and homing of AML cells in NOD/SCID mice is mediated by at least 2 cooperative pathways: ADCC caused by NK and/or other CD122-dependent cells; and, specific inhibitory effects of 7G3 blocking IL-3/CD123 signaling pathways.

TABLE 1
Ex vivo effectiveness of 7G3 treatment on human normal and leukemia cells
is associated with CD123 expression on CD34+/CD38− cells.

| | | | | | | Engraftment of |
|---|---|---|---|---|---|---|
| | | | CD34+/CD38− | CD34+ | | 7G3 treated |
| | | CD34+ | CD123 | CD123+ | CD123 | cells^{b} |
| Cells | AML | CD38− | Expression | expression | Expression | (engraftment as |
| Transplanted | subtype^{a} | (%) | (RFI) | (RFI) | (RFI) | % control) |
| AML | | | | | | |
| 1 | M0 | 2.9 | 52.1 | 85.3 | 76.3 | 3.6 |
| 2 | M1 | 2.2 | 26.1 | 20.8 | 34.6 | 5.3 |
| 3 | M5b | 0.048 | ^{c}9.9 | ^{c}27.4 | 29.4 | 19.5 |
| 4 | M5^{a} | 3.5 | 36.5 | 47.0 | 18.8 | 6.6 |
| 5 | M2 | 6.2 | 13.8 | 12.2 | 11.2 | 97.1 |
| 6 | M2 | 0.18 | ^{c}51.7 | ^{c}52.4 | 21.8 | NE |
| 7 | M5b | 0.010 | ^{c}20 | ^{c}86 | 54.3 | NE |
| 8 | M4eo | 4.9 | 24.2 | 18.6 | 16.7 | 1.5 |
| Normal Cells | | | | | | |
| NBM-1 | NA | 0.42 | 12.0 | 17.1 | 3.0 | 139.9 |
| NBM-2 | NA | 2.3 | 6.7 | 8.8 | 3.0 | 34.8 |
| NBM-3 (CD34+) | NA | 0.40 | 7.0 | 6.5 | 6.5 | 50.4 |

CD34, CD38 and CD123 antigens were stained with fluorochrome-conjugated antibodies. The CD123 expression on specific subpopulations and the entire sample of the original AML patient or NBM donor, based on CD34 and CD38 expression was measured as the relative fluorescence index (RFI) determined from the ratio of the geometric mean of the fluorescence intensity of the stained sample to isotype control. NBM-3 was a CD34+ sorted normal bone marrow sample. High CD123 expression is associated with a decrease in engraftment of 7G3 treated cells.
^{a}FAB criteria
^{b}The engraftment of 7G3 treated cells is expressed as mean engraftment in the 7G3 ex vivo incubated j group as a percentage of the mean engraftment level in IgG2a incubated group based on FIG. 1.
^{c}Sample had very low CD34 expression or number of CD34+ cells
NE = no engraftment in controls
NA = not applicable

### Discussion

The consistent overexpression of CD123 on AML blasts and LSCs provides a promising therapeutic target for the treatment of AML either alone or in combination with established therapies, especially for relapse or minimal residual disease. Several therapeutics based on CD123 have been devised and have demonstrated anti-AML effects in various assays^{23,47-49}. In the current study, 7G3 has been demonstrated to specifically and consistently inhibit IL-3 mediated signaling pathways and subsequent induced proliferation of different AML samples in vitro. Moreover, 7G3 treatment profoundly reduced AML-LSC engraftment and improved mouse survival. Mice with pre-established disease showed reduced AML burden in the BM and periphery and impaired secondary transplantation upon treatment establishing that AML-LSCs in treated mice were directly targeted. These results provide clear validation for therapeutic anti-CD123 monoclonal antibody targeting of AML-LSCs, and for translation of in vivo preclinical research findings towards a potential clinical application.

### Example 2

CSL360 is a chimeric antibody obtained by grafting the light variable and heavy variable regions of the mouse monoclonal antibody 7G3 onto a human IgG constant region. Like 7G3, CSL360 binds to CD123 (human IL-3Rα) with high affinity, competes with IL-3 for binding to the receptor and blocks its biological activities.³³ The mostly human chimeric antibody CSL360, can thus potentially also be used to target and eliminate AML LSC cells. CSL360 also has the advantage of potential utility as a human therapeutic agent by virtue of its human IgG1 Fc region which would be able to initiate effector activity in a human setting Moreover, it is likely that in humans it would show reduced clearance relative to the mouse 7G3 equivalent and be less likely to be immunogenic. The mechanisms of action of CSL360 in treatment of CD123 expressing leukemias may involve 1) inhibition of IL-3 signalling by blocking IL-3 from binding to its receptor, 2) recruitment of complement after the antibody has bound to a target cell and cause complement-dependent cytotoxicity (CDC), or 3) recruitment of effector cells after the antibody has bound to a target cell and cause antibody dependent cell cytotoxicity (ADCC).

Methods developed to study antibody dependent cell cytotoxicity (ADCC) are described below, and can be categorised into methods which analyse (1) target cell population or (2) effector cell population in the assay. Methods involved with analysis of target cells measure target cell lysis or early apoptosis of target cells brought about by ADCC. Methods that examine the effector population measure induction of membrane granules on effector cells such as NK cells as a marker for NK cell-induced cell lysis.

### Methods

Measuring ADCC Using a⁵¹Chromium Release Assay

The murine lymphoid cell line CTL-EN engineered to express CD123 as described by Jenkins et al⁵⁰ or freshly thawed leukemic cells (5×10⁶) were incubated with 250 μCi of⁵¹Cr-sodium chromate for one hour at 37° C. Cells were washed three times with RPMI-10% FCS medium to remove any free ⁵¹Cr-sodium chromate. Chromium labelled target cells were dispensed at 10,000 cells/well in round bottom 96-well plates. CSL360 or an isotype control antibody, (MonoRho, recombinant anti-Rhesus D human immunoglobulin G1), was added at 10 μg/mL.

Freshly isolated PBMC were added as effector cells at different ratios in triplicates and incubated for four hours at 37° C. in a 5% CO₂ incubator. Total sample volume was 200 μL/well. After the incubation period, plates were centrifuged for 5 minutes at 600×g, 100 μL of supernatant removed and ⁵¹Cr released measured in a Wallace γ-counter.

Specific lysis was determined by using the formula, % lysis=100×[(mean cpm with antibody−mean spontaneous cpm)/(mean maximum cpm−mean spontaneous cpm)]. Spontaneous release was obtained from samples that had target cells with no antibody and no effector cells. Maximum release was determined from target cells treated with 1% (v/v) Triton X-100.

### Measuring ADCC Using a Calcein AM-Labelled Target Cell Assay

ADCC induced by CSL360 was measured by the method described by Neri et al⁵². This method involved labelling of target cells with Calcein AM instead of ⁵¹Chromium. Target cells were incubated with 10M Calcein AM (Invitrogen, cat. no. C3099) for 30 minutes at 37° C. in a 5% CO₂ incubator. Labelled cells were washed to remove any free Calcein AM and then dispensed in round bottom plates at 5000 cells per well. Effector cells were added at different ratios. Relevant antibodies were added to a final concentration of 10 μg/mL, cells with no antibody serving as negative controls. Plates were incubated for 4 hours at 37° C. in a 5% CO₂ incubator. After the incubation period, plates were centrifuged at 600×g for 5 minutes. 100 μL of supernatant was removed and fluorescence measured in an Envision microplate reader (excitation filter 485 nm, emission filter 535 nm). Specific lysis was calculated by using the formula, % lysis=100×[(mean fluorescence with antibody−mean spontaneous fluorescence)/(mean maximum fluorescence−mean spontaneous fluorescence)]. Maximum fluorescence was determined by the lysis of cells with 3% Extran and spontaneous lysis was the fluorescence obtained with target cells without any antibody or effector cells.

### Measuring ADCC as Effector Cell Expression of Membrane Granule Protein CD107a as a Surrogate Marker of Cytolysis

Fischer et al⁵¹ demonstrated that expression levels of CD107a, a membrane-associated lytic granule protein, by NK cells correlates with target cell cytotoxicity. This method was used to assess ADCC activity of CSL360. The method involved incubation of freshly isolated human PBMC from a buffy coat with target cells. Target cells used were either CD123-expressing cell lines or primary human AML cells. Target cells were added to human PBMC at 1:1 ratio in presence or absence of antibody. Nonspecific or spontaneous expression of CD107a was assessed with human PBMC without any antibody or target cells added. PE-Cy5 conjugated CD107a monoclonal antibody (BD Pharmingen, cat. no. 555802) was added to all samples and cells were incubated for three hours at 37° C. in a 5% CO₂ incubator. After the first hour of incubation, Brefeldin A (BFA) was added. At the end of incubation, cells were washed and stained with anti-CD56-PE (BD Pharmingen, cat. no. 347747) and anti-CD16-FITC (BD Pharmingen, cat. no. 555406) monoclonal antibodies. Cells were then analysed by flow cytometry using a FACS Calibur and analysed (Flow Jo Software Tree Star, Inc.) for CD56dimCD16+CD107a cells that represent NK cells expressing FcRγIIIA receptor that have expressed the membrane associated lytic granule protein.

### Results

### CSL360 Induces ADCC in an AML Sample and a CD123-Expressing Cell Line as Assessed by a ⁵¹Chromium-Release Assay

Total uptake of ⁵¹Chromium by CTLEN cells were between 2000-1500cpm as compared to only about 400-200cpm by AML cells as determined by maximum chromium release with detergent lysis. 15% lysis of AML (SL) cells was observed with CSL360 at 100:1 ratio of effector to target cells compared to 1.9% lysis with negative control antibody, MonoRho. 51% lysis of CTLEN cells was observed with CSL360 at 100:1 ratio of effector to target cells compared to 5% lysis with negative control antibody MonoRho (Table 2). These results suggested that CTLEN cells were more susceptible to CSL360-mediated ADCC lysis than the AML cells even though AML cells had higher levels of surface expression of CD123.

### CSL360 Induces ADCC in AML Samples and CD123-Expressing Cell Lines as Assessed by CD107a Expression on Effector NK Cells

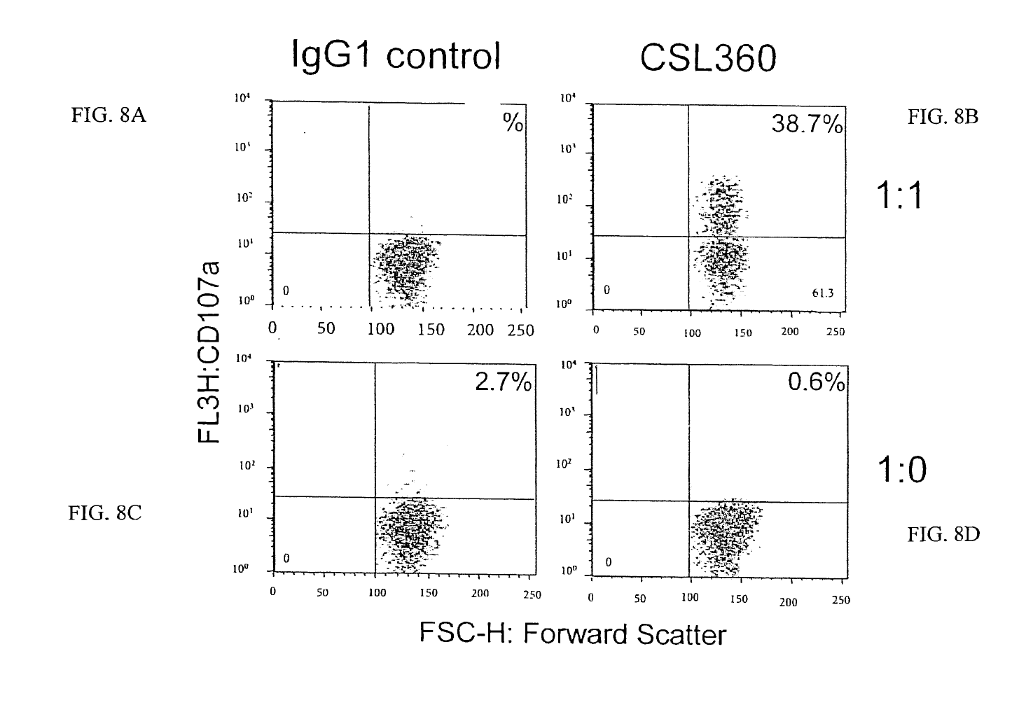

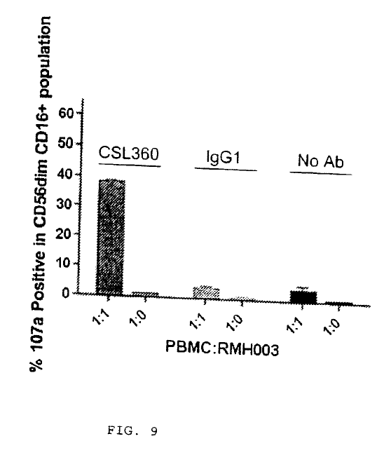

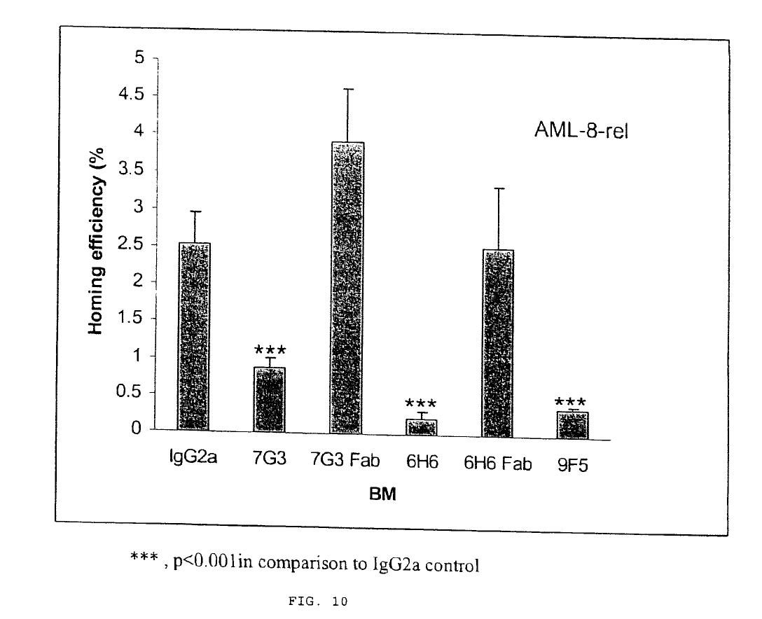

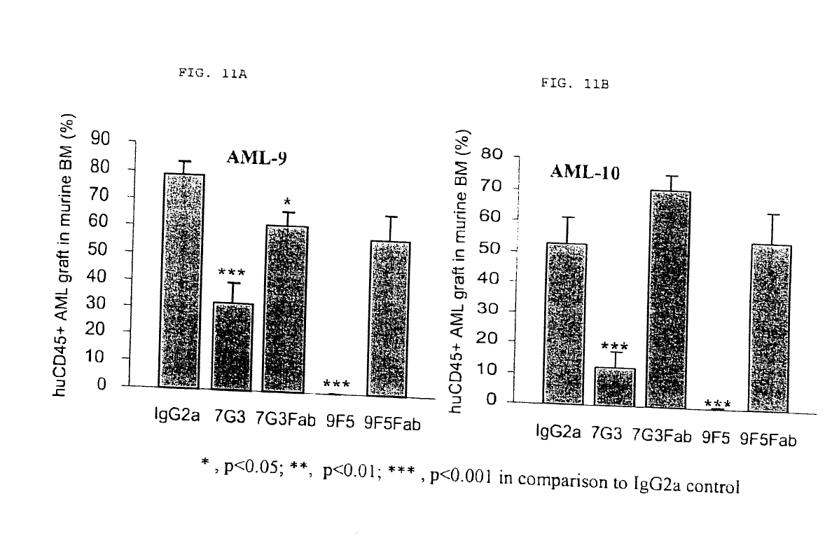

FIG. 8 shows flow cytometer analyses demonstrating the induction of membrane lytic granule, CD107a on NK cells derived from mixing PBMC from a normal donor incubated with an AML patient sample, RMH003 in the presence of CSL360 or isotype control antibody. NK cells within this mixed population were gated from lymphocyte populations that expressed CD56 (NK marker) and CD16 (FcRγIIIA). The data show that NK exposed to AML cells coated with CSL360 demonstrated significantly elevated CD107a (˜39% CD107a positive cells in FIG. 8B) compared to NK from the same donor and patient samples incubated with isotype control antibody (˜3% CD107a positive cells in FIG. 8A). Induction of CD107a on the donor NK cells is target cell-dependent since CD107a was not detected if CSL360 was added to effector cells in the absence of the target AML patient cells (FIG. 8D). FIG. 9 shows data from the same experiment plotted as a histogram.

Data generated in a similar way as above from a number of cell lines engineered to express human CD123 (CTLEN, EL4) or human leukemic cell line expressing endogenous CD123 (TF-1) and primary samples from leukemic patients as target cells incubated with effector cells derived from up to 3 different donors are included in Table 3. The data are expressed as percentages of NK cells that expressed CD107a incubated with different samples in presence of CSL360 or without added antibody. Two mouse cell lines expressing human CD123 induced CD107a expression in NK cells in presence of CSL360. 4/8 primary leukemic samples demonstrated CSL360-mediated expression of CD107a on NK cells. RMH007 induced expression of CD107a in NK cells even in absence of CSL360. RBH013 gave similar results with PBMC from one donor, however, with a different donor CD107a expression was specific to CSL360 indicating donor-specific susceptibility to NK-mediated ADCC induced by CSL360 in this case.

Six of the eight primary leukemic samples were examined for ADCC effects with different donors as a source for effector cells. An important observation was that samples that were susceptible to ADCC usually induced CD107a in effector cells irrespective of the donor. Similarly, samples that were resistant to ADCC also generally remained negative irrespective of donor cells.

### CSL360 Induces ADCC in AML Samples as Assessed by a Calcein-AM Release Assay

Calcein released in the medium by lysed cells is an indicator of ADCC-mediated cell lysis. Patients RMH003 and RMH008 showed susceptibility to ADCC in this assay whereas RMH009, RMH010 and RBH013 appeared resistant to lysis (Table 4). All of these five patients were tested for their susceptibility to CSL360-mediated ADCC in a NK cell CD107a expression assay with same effector cells as used for this assay and comparative results are shown in Table 5. Status of ADCC in three out of six patients samples were in agreement with the two different assays.

TABLE 2
ADCC mediated lysis in ⁵¹Chromium release assay

| | % Lysis with CSL360 at E:T | | % Lysis with MonoRho at E:T | |
|---|---|---|---|---|
| Sample | 100:1 | 10:1 | 100:1 | 10:1 |
| CTLEN | 51 | 10 | 5 | 1 |
| SL | 15 | 2.4 | 1.9 | 4.5 |
| (AML) | | | | |

TABLE 3
Surface expression of CD107a as a measure of ADCC activity.

| | % of NK cells expressing CD107a | | | |
|---|---|---|---|---|
| | CSL360 at E:T | | No Antibody at E:T | |
| Sample | 1:1 | 1:0 | 1:1 | 1:0 |
| CTLEN-001 | 6.7 | 0.7 | 0.6 | 0.6 |
| CTLEN-008 | 24 | 1.0 | 2.6 | 0.6 |
| CTLEN-010 | 5.7 | 2.2 | 2.7 | — |
| CTLEN-011-PBMC-1 | 8.1 | 0.8 | 2.7 | 0.6 |
| CTLEN-011-PBMC-2 | 10.5 | 1.3 | 0.25 | 0.28 |
| EL4hi/lo-001 | 2.8 | 0.8 | 0.6 | 0.6 |
| EL4hi-002 | 2.9 | 0.7 | 0.5 | 0.5 |
| EL4hi-003 | 15.0 | 1.1 | 6.7 | 0.6 |
| EL4hi-004 | 16 | 0.46 | 0.04 | — |
| EL4hi-005 | 21 | 1.5 | 7.0 | 0.6 |
| EL4hi-010 | 34 | 1.2 | 2 | 2 |
| EL4hi-011-PBMC-l | 15.5 | 0.18 | 5.7 | 0.45 |
| EL4hi-011-PBMC-2 | 23.5 | 0.6 | 0.12 | 0.18 |
| TF-1(IL-3) | 1.45 | 0.7 | 1.4 | 0.5 |
| TF-1 (GM-CSF) | 0.7 | 0.7 | 0.6 | 0.5 |
| RMH003^{a} (AML) | 30 | nd | 3.5 | nd |
| RMH003^{b} (AML) | 33 | 1.4 | 0.8 | 0.6 |
| RMH003^{c} (AML) | 38.0 | 1.2 | 3.7 | 0.4 |
| RMH007 (B-ALL) | 16.4 | 8.9 | 16.7 | 4.0 |
| RMH008^{a} (AML) | 9 | nd | 4 | nd |
| RMH008^{b} (AML) | 16 | 1.8 | 1.6 | 0.6 |
| RMH008^{c} (AML) | 8.7 | 1 | 0.9 | 0.4 |
| RMH009^{a} (B-ALL) | 3.0 | nd | 1.8 | nd |
| RMH009^{b} (B-ALL) | 1.7 | 1.0 | 0.9 | 0.55 |
| RMH009^{c} (B-ALL) | 7.4 | 8.6 | 4.2 | 3.6 |
| RMH010^{a} (AML) | 14 | nd | 6 | nd |
| RMH010^{b} (AML) | 25.5 | 7.8 | 5.2 | 3.6 |
| RMH011 (AML) | 6.6 | 6.6 | 3.4 | 2.2 |
| RBH009^{a} (AML) | 8.5 | nd | 4.5 | nd |
| RBH009^{b} (AML) | 8.0 | 6.7 | 3.8 | 2.8 |
| RBH013^{a} (AML) | 10.0 | nd | 10.0 | nd |
| RBH013^{b} (AML) | 22.0 | 5.9 | 3.3 | 2.7 |

^{a,b,c}indicate that samples were tested for ADCC with different donors as a source for effector cells.

TABLE 4
Assessment of ADCC in Calcein release assay.

| % Lysis | |
|---|---|
| CSL360 at E:T | No antibody at E:T |

| Sample | 100:1 | 10:1 | 100:1 | 10:1 |
|---|---|---|---|---|
| RMH003 (AML) | 82.6 | 39.2 | 19.9 | 7.1 |
| RMH008 (AML) | 100 | 59.5 | 58.2 | 0 |
| RMH009 (B-ALL) | 0 | 0 | 0 | 0 |
| RMH009* (B-ALL) | 0 | 0 | 0 | 0 |
| RMH010 (AML) | 0 | 4 | 4 | 8 |
| RBH013 (AML) | 0 | 0 | 0 | 0 |

*Repeat assay with sample RMH009 using another source of PBMC as effector cells.

TABLE 5
Comparison of Flow cytometry based assays to lysis assays.

| | | | ⁵¹Chromium |
|---|---|---|---|
| Samples | CD 107a Status | Calcein release | release |
| CTLEN | Positive | nd | Positive |
| EL4hi | Positive | nd | nd |
| RMH003 ^{a} (AML) | Positive | Positive | nd |
| RMH008 ^{a} (AML) | Positive | Positive | nd |
| RMH007 (B-ALL) | Negative | nd | nd |
| RMH009 ^{a} (B-ALL) | Negative | Negative | nd |
| RMH010 ^{a} (AML) | Positive | Negative | nd |
| RBH013 ^{a} (AML) | Positive | Negative | nd |
| SL (AML) | Nd | nd | Positive |

^{a} These samples were tested for ADCC using CD107a and Calcein release assays with same effector cells for both assays.
* not done

### Discussion

Through the use of several assays all acknowledged to measure ADCC activity, albeit with varying sensitivity, it has been shown that CSL360 can induce ADCC responses in mouse cell lines maintained in culture that express ectopic human CD123. Importantly, CSL360 also was able to induce an ADCC response against primary human AML patient samples in the presence of functional effector cells from normal donors. This data suggests that in some leukemic patients whose leukemic cells including LSC, express sufficient levels of CD123 that CSL360 administered therapeutically may be able to induce ADCC-directed elimination of the leukemic cells particularly if the patients retained some functional effector cells in their circulation, for example such as those in remission or with minimal residual disease.

### Example 3

The ubiquitous expression of CD123 on AML cells including LSC and the evidence implicating IL-3 having an important role in the etiology of AML suggested that the ability to block IL-3Rα function would be critical for any therapeutic activity of an antibody targeting IL-3Rα such as 7G3. In this example, it is demonstrated somewhat surprisingly, that the ability of 7G3 to inhibit the engraftment or repopulation of NOD/SCID mice by AML patient samples is at least partially dependent upon the effector function responses elicited by the Fc domain of 7G3. Also, other IL-3Rα antibodies that do not significantly inhibit IL-3Rα function also block engraftment and hence demonstrate therapeutic activity in the NOD/SCID mouse model of AML.

### Methods

### F(ab)′2 Fragment Preparation

F(ab)′2 fragments for 6H6, 9F5 and 7G3 were derived by pepsin cleavage using immobilised pepsin-agarose (22.5 U pepsin agarose/mg antibody) incubated with antibody at 37° C. for 2 hr. Digestion was quenched by pH adjustment using 3M Tris to 6.5. Immobilised beads were separated from resultant F(ab)′2 by centrifugation. F(ab)′2 of 7G3 was purified from residual immuunoglobulin and other contaminants using tandem chromatographic procedures: thiophilic adsorption chromatography (20-0% ammonium sulphate gradient in 40 mM HEPES over 15 column volumes) and anion exchange chromatography. 9F5 F(ab)′2 and 6H6 F(ab)′2 were purified by ion exchange chromatography followed by affinity chromatography. Endotoxin levels were quantitated by LAL chromogenic assay. Where endotoxin levels were >10 EU/mL, Detoxigel was used to reduce endotoxin levels. 7G3 F(ab)′2 as expected, retained CD123-neutralising activity as assessed by the IL-3-dependent TF-1 proliferation assay (data not shown).

### AML Patient Samples

Peripheral blood cells were collected from 3 newly diagnosed patients after informed consent was obtained. AML patients were diagnosed and classified according to the French-American-British (FAB) criteria. AML-8-rel was originally classified as M4 at first diagnosis, AML-9 was classified as M5a, and AML-10 was unclassified. AML blasts were isolated by Ficoll density gradient centrifugation and frozen in aliquots in liquid nitrogen.

### In Vitro Antibody Treatment

Monoclonal antibodies against IL-3 receptor α chain (CD123), 7G3, 9F5, 6H6 and their F(ab)′2 fragments, were used to treat the cells harvested from AML patients. IgG2a was used in parallel as a control. Thawed AML cells were seeded in XVIVO10 plus 15% BIT and independently incubated with antibodies at the concentration of 10 μg/mL. After 2 hours of incubation at 37° C., harvested leukemic cells were intravenously injected into sub-lethally irradiated NOD/SCID mice for repopulating assays.

Xenotransplantion of Human Cells into NOD/SCID Mice

Xenotransplantion was performed essentially performed as outlined in Example 1. NOD/SCID mice were bred and housed at the Animal facility of the University Health Network/Princess Margaret Hospital. Animal studies were performed under the institutional guidelines approved by the University Health Network/Princess Margaret Hospital Animal Care Committee. Transplantation of leukemic cells into NOD/SCID mice was performed as previously described³. Briefly, all mice in the same experiment were irradiated at the same time with the dose of 300cGy before being injected with an equal number of human cells. For intravenous transplantation, 5 mice were used for each group with injection of 5-10 million leukemic cells per mouse. Engraftment levels of human AML were evaluated based on the percentage of CD45+ cells by flow cytometry of the murine bone marrow.

### Cell Staining and Flow Cytometry

Cells from the bone marrow of treated mice were stained with mouse antibody specific to human CD45 (anti-CD45) conjugated to APC (Beckman-Coulter), anti-CD34 conjugated to fluorescein isothiocyanate (FITC), and anti-CD38-PC5 (Becton-Dickinson). Isotypic controls were used to avoid false positive cells. Anti-CD123-PE (clone 9F5 and 7G3, Becton-Dickinson) was used to test the expression of IL-3 receptor α chain on the AML cells. Stained cells were analyzed using Caliber (Becton-Dickinson).

### Statistical Analysis

Data are presented as the mean±s.e.m. The significance of the differences between treated groups was determined by p value using Student's t-test. Results were considered statistically significant at P<0.05.

### Results

### Anti-IL-3Rα Antibody Fc Domain Contributes Significantly to Inhibit AML Homing Capacity

The data in Example 1, FIGS. 5a and b indicate that ADCC caused by NK and/or other CD122-dependent cells contributes to the ability of 7G3 to inhibit homing and repopulation of AML cells into the bone marrow of NOD/SCID mice and is in addition to effects of 7G3 blocking IL-3/CD123 signaling pathways. To examine this directly, the effect of other poorly-neutralising anti-IL-3Rα antibodies 6H6 and 9F5 on the homing of an AML sample treated ex vivo was examined. Both 6H6 and 9F5 specifically bind CD123 however, unlike 7G3 they do not block IL-3Rα function³³. This is also evident in FIG. 6a which shows that unlike 7G3, both 9F5 and 6H6 failed to inhibit IL-3-induced signaling including CD131 (βc) tyrosine phosphorylation, STAT-5 phosphorylation and Akt phosphorylation even at the highest doses tested. FIG. 10 shows that 6H6 and 9F5 nevertheless, potently inhibited homing of AML cells to the BM at least as well as 7G3 in this experiment.

The contribution of the Fc domain for the effects of 7G3 for inhibition of homing was assessed by testing F(ab)′2 fragments of both 7G3 and 6H6. Antibody F(ab)′2 fragments lack the Fc effector immunoglobulin domain and are not able to elicit ADCC or CDC responses. FIG. 10 also shows that the F(ab)′2 fragments of both 7G3 and 6H6 did not inhibit AML cell homing in this experiment and indicate that the Fc domain of both antibodies is important for the inhibition of homing of AML cells to the bone marrow.

### Anti-IL-3Rα Antibody Fe Domain Contributes Significantly to Inhibit Bone Marrow Engraftment and Repopulation Capacity of AML Cells

The experiment was then extended to evaluate the contribution of IL-3Rα neutralisation and effector activity for the inhibition of engraftment of AML cells into the bone marrow of recipient mice. Two AML patient samples were treated ex vivo with the various intact antibodies and antibody fragments at a concentration of 10 μg/mL at 37° C. for 2 hours. Following incubation, cells were centrifuged to remove unbound antibodies and transplanted to sub-lethally irradiated NOD/SCID mice. The engraftment levels of human AML were analyzed by assessing the percentage of huCD45 positive cells in the bone marrow of the mice 4 weeks post-transplantation. As shown in FIGS. 11a and b, 7G3 as expected, significantly inhibited the engraftment into NOD/SCID mice of both AML patient samples. Consistent with the effect on homing, 9F5 also potently inhibited AML cell engraftment of both patient samples. Interestingly, FIG. 11a shows that for patient sample AML-9 the F(ab)′2 fragments of both 7G3 and 9F5 demonstrated significantly reduced inhibitory capacity, but did not completely allow engraftment to return to the levels seen with control antibody. In contrast, for sample AML-10 there was no inhibitory effects of both F(ab)′2 fragments.

### Discussion

Taken together, these results indicate that in addition to the ability of 7G3 to neutralise IL-3Rα function, that the Fc domain of 7G3 is also important for inhibition of the homing and engraftment capacities of AML cells. Without the Fc domain, antibodies against CD123 significantly lose their capacity to inhibit homing, lodgement, and repopulation of AML-LSCs in NOD/SCID mice.

### Example 4

A number of methods have been described for increasing the effector function activity of antibodies. These methods can include amino acid modification of the Fc region of the antibody to enhance its interaction with relevant Fc receptors and increase its potential to facilitate antibody-dependent cell-mediated cytotoxicity (ADCC) and antibody-dependent cell-mediated phagocytosis (ADCP)^{34,35}. Enhancements in ADCC activity have also been described following the modification of the oligosaccharide covalently attached to IgG1 antibodies at the conserved Asn²⁹⁷ in the Fc region³⁴. In a further study³⁶ the expression of human IgG1 antibodies in Lec13 cells, a variant Chinese hamster ovary cell line which is deficient in its ability to add fucose to an otherwise normal oligosaccharide, resulted in a fucose-deficient antibody with up to 50-fold improved binding to human FcγRIIIA and improved ADCC activity.

Alternative approaches to producing defucosylated antibodies have also been described through culturing antibody-expressing cells in the presence of certain glycosidase inhibitors⁵³. In this study, CHO cells expressing antibodies of interest were cultured in the presence of kifunensine, a potent α-mannosidase I inhibitor, which resulted in secretion of IgGs with oligomannose-type glycans that do not contain fucose. These antibodies exhibited increased affinity for FcR and enhanced ADCC activity.

In this example, generation and testing of CSL360 variants with enhanced ADCC activity through Fc-engineering or defucosylation is described.

### Methods

### Mammalian Expression Vector Construction for Transient Expression of CSL360 and Fc Optimized CSL360

The genes for both the light and heavy chain variable region of the murine anti-CD123 antibody 7G3 were cloned from total 7G3.1B8 hybridoma RNA isolated using the NucleoSpin RNA II kit (BD Bioscience) according to the manufacturer's instructions. First-strand cDNA was synthesized using the SMART RACE Amplification kit (Clontech) and the variable regions amplified by RACE-PCR using proof-reading DNA polymerase, Plantinum® Pfx DNA polymerase (Invitrogen). The primers used for the variable heavy region were UPM (Universal Primer A mix, DB Bioscience) and MH2a (5′AATAACCCTTGACCAGGCATCCTA3′ (SEQ ID NO: 1)). Similarly, the variable light region was amplified using UPM and MK (5′CTGAGGCACCTCCAGATGTTAACT3′ SEQ ID NO: 2)). Using standard molecular biology techniques, the heavy chain variable region was cloned into either; a) the mammalian expression vector pcDNA3.1(+)-hIgG1, which is based on the pcDNA3.1(+) expression vector (Invitrogen) modified to include the human IgG1 constant region or, b) pcDNA3.1(+)-hIgG1_{S239D/A330L/1332E}, or c) pcDNA3.1(+)-hIgG1_{S239D/I332E}. The vectors used in b) and c) encode for protein that incorporate amino acid mutations which are reported to result in an antibody with significantly improved ADCC activity³⁵. These mutations were introduced using QuikChange mutagenesis techniques (Stratagene). The light chain variable region was cloned into the expression vector pcDNA3.1(+)-hκ, which is based on the pcDNA3.1(+) expression vector modified to include the human kappa constant region.

### Cell Culture

FreeStyle™ 293-F cells were obtained from Invitrogen. Cells were cultured in FreeStyle™ Expression Medium (Invitrogen) supplemented with penicillin/streptomycin/fungizone reagent (Invitrogen). Prior to transfection the cells were maintained at 37° C. with an atmosphere of 8% CO₂.

### Transient Transfection

Transient transfections of the expression plasmids using FreeStyle™ 293-F cells were performed using 293 fectin transfection reagent (Invitrogen) according to the manufacturer's instructions. The light and heavy chain expression vectors were combined and co-transfected into the FreeStyle™ 293-F cells. Cells (1000 ml) were transfected at a final concentration of 1×10⁶ viable cells/mL and incubated in a Cellbag 2L (Wave Biotech/GE Healthcare) for 5 days at 37° C. with an atmosphere of 8% CO₂ on a 2/10 Wave Bioreactor system 2/10 or 20/50 (Wave Biotech/GE Healthcare). Pluronic® F-68 (Invitrogen), to a final concentration of 0.1% v/v, was added 4 hours post-transfection. 24 hours post-transfection the cell cultures were supplemented with Tryptone N1 (Organotechnie, France) to a final concentration of 0.5% v/v. The cell culture supernatants were then harvested by filtration through a Millistak+ POD filter (Millipore) prior to purification.

### Kifunensine Treatment

For production of defucosylated antibodies where indicated kifunensine (Toronto Research Chemicals) was added to the culture medium of transiently transfected FreeStyle™ 293-F cells (24 hours post transfection) to a final concentration of 0.5 μg/mL as described⁵³.

### Analysis of Protein Expression

After 5 days 20 μl of culture supernatant was electrophoresed on a 4-20% Tris-Glycine SDS polyacrylamide gel and the antibody was visualised by staining with Coomassie Blue reagent.

### Antibody Purification

In addition to the chimeric CSL360 described in Example 2, in this example the use of a humanised variant of CSL360 (hCSL360) is also described. This was produced by standard CDR grafting techniques where the murine CDR regions from 7G3 were grafted on suitable human variable framework regions⁵⁴. The resulting humanised antibody contains entirely human framework sequence. As a result of the humanisation process, the MAb affinity for CD123 was moderately decreased (indicative KD's of 1.06 nM vs 12.8 nM for CSL360 and hCSL360 respectively) however, the binding specificity remained unchanged and the hCSL360 retained potent CD123-neutralisation activity as measured by IL-3-dependent TF-1 cell proliferation (indicative IC₅₀'s of 5 nM vs 19 nM for CSL360 and hCSL360 respectively). Affinity optimisation was employed using standard ribosome display-based mutagenesis⁵⁵ to restore the binding affinity of hCSL360 to levels at least equivalent to the parent mouse MAb 7G3 and the chimeric CSL360. An affinity optimised MAb clone was produced (168-26) that exhibited comparable CD123 binding affinity and neutralisation of CD123 activity to the parent MAb (indicative KD of 0.6 nM for binding to CD123 and IL-3 neutralisation IC₅₀ of 6 nM). Fc engineered derivatives of this clone containing the IgG1 Fc domains with the three amino acid substitutions S239D/A330L/I332E (168-26Fc3) or with the two amino acid substitutions S239D/I332E (168-26Fc2) were also produced as described above for hCSL360.

The unmodified chimeric CSL360, humanised variant (hCSL360) and the ADCC-optimised and humanised CSL360_{S239D/I332E} (hCSL360Fc2) and CSL360_{S239D/A330L/I332E} (hCSL360Fc3) and material derived from kifunensine-treated cells were purified using protein A affinity chromatography at 4° C., with MabSelect resin (5 ml, GE Healthcare, UK) packed into a 30 mL Poly-Prep empty column (Bio-Rad, CA). The resin was first washed with 10 column volumes of pyrogen free GIBCO Distilled Water (Invitrogen, CA) to remove storage ethanol and then equilibrated with 5 column volumes of pyrogen free phosphate buffered saline (PBS) (GIBCO PBS, Invitrogen, CA). The filtered conditioned cell culture media (1 L) was then loaded onto the resin by gravity feed. The resin was then washed with 5 column volumes of pyrogen free PBS to remove non-specific proteins. The bound antibody was eluted with 2 column volumes of 0.1M glycine pH 2.8 (Sigma, Mo.) into a fraction containing 0.2 column volumes of 2M Tris-HCl pH 8.0 (Sigma, Mo.) to neutralise the low pH. The eluted antibody was dialysed for 18 hrs at 4° C. in a 12 ml Slide-A-Lyzer cassette MW cutoff 3.5 kD (Pierce, Ill.) against 5 L PBS. The antibody concentration was determined by measuring the absorbance at 280 nm using an Ultraspec 3000 (GE Healthcare, UK) spectrophotometer. The purity of the antibody was analysed by SDS-PAGE, where 2 μg protein in reducing Sample Buffer (Invitrogen, CA) was loaded onto a Novex 10-20% Tris Glycine Gel (Invitrogen, CA) and a constant voltage of 150V applied for 90 minutes in an XCell SureLock Mini-Cell (Invitrogen, CA) with Tris Glycine SDS running buffer before visualised using Coomassie Stain, as per the manufacturer's instructions.

### Results

### ADCC Testing of Wildtype CSL360 and the Fe-Engineered CSL360 Variants

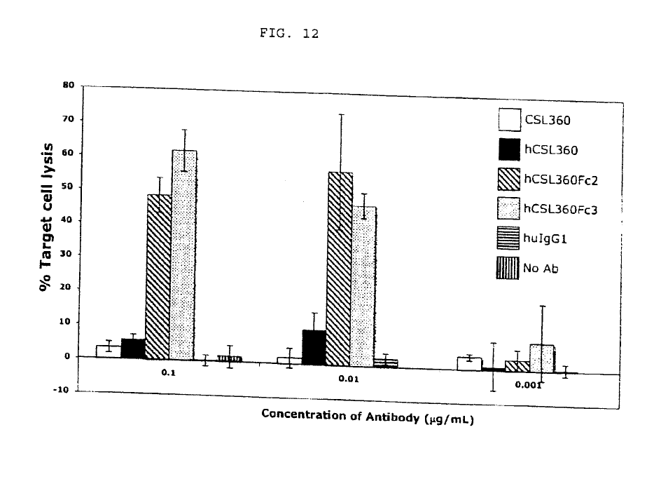

To test the effector activity of the various variant antibodies the CD123-expressing CTLEN cell line was used as a target cell line and ADCC activity assessed using the calcein AM release assay as outlined in Example 2 in the presence of normal PBMC as a source of effecter cells. FIG. 12 shows the comparison of chimeric CSL360 and a humanised variant (hCSL360) antibody as well as the Fc-modified variants hCSL360Fc2 and hCSL360Fc3 for their abilities to induce ADCC-directed lysis of the CTLEN target cell line. The data show that both the chimeric CSL360 as well as the humanised variant without modification of the Fc domain had a detectable but modest ability to induce ADCC against the CTLEN cell line (5-10% target cell lysis) and is consistent with the findings outlined in example 2. Both variants with modified Fc domains, hCSL360Fc2 and hCSL360Fc3, demonstrated significantly enhanced capacity to elicit ADCC-directed lysis of the CTLEN target cells with 50-60% target cell lysis being observed when tested at the same concentration as the Fc unmodified antibodies.

### Testing of CSL360, Fc-Engineered CSL360 Variants and Defucosylated CSL360 for Binding to Fc Receptors

As already mentioned, antibody Fc effector function is mediated through binding to Fc gamma receptors (FcγR) expressed on the various effector cells of the innate immune system³⁷.

### Optimisation of Antibodies for Enhanced Binding to FcγR's Results in Greater Effector Cell Activation and Greater Killing of Antibody-Coated Tumor Cells.

The relative affinities of the various human FcγR's for hCSL360, the Fc engineered variants hCSL360Fc2 and hCSL360Fc3 and defucosylated hCSL360 produced by kifunensine treatment (hCSL360kif) were measured with a BIAcore A100 biosensor. The various antibodies were individually captured on a CM5 BIAcore chip coupled with CD123. Soluble FcγR's (huFcγRI, huFcγRIIb/c and huFcγRIIIa (obtained from R & D Systems) at concentrations ranging from 0.3 nM to 800 nM were flowed over the respective surfaces and affinity measurements determined by fitting the data to kinetic and/or steady state models.

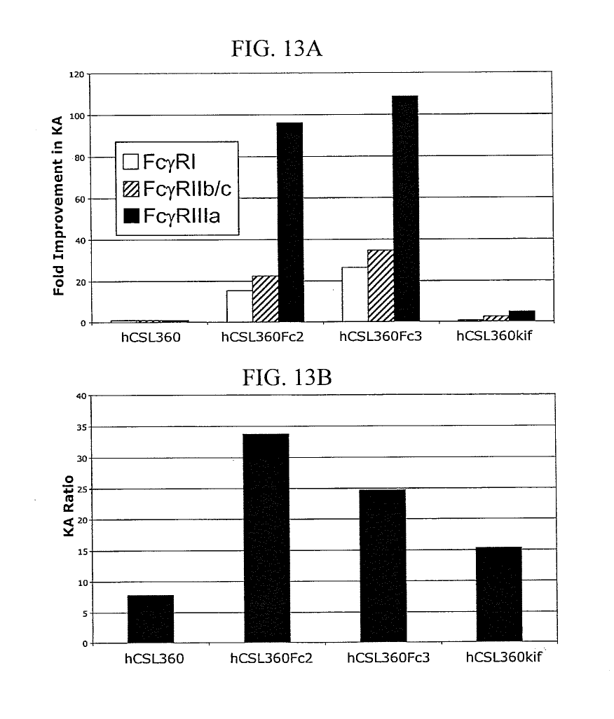

FIG. 13A compares the affinities (KA) of hCSL360Fc2, hCSL360Fc3 and hCSL360kif relative to hCSL360 for binding to huFcγRI, huFcγRIIb/c and huFcγRIIIa. The results are broadly similar for hCSL360Fc2 and hCSL360Fc3 with an approximate 15-35-fold increase in KA relative to hCSL360 for binding to huFcγRI and huFcγRIIb/c. The most pronounced increase in binding was seen for huFcγRIIIa where affinities were increased ˜100-fold. Although the absolute increase in fold affinity of hCSL360kif was lower than the Fc-engineered variants, a similar pattern was observed with huFcγRIIIa once again exhibiting the greatest fold improvement (˜5-fold) compared to huFcγRI (0.75-fold) and huFcγRIIb/c (2.6-fold).

Recent studies have shown that rather than absolute affinities, a high activating/inhibitory (A/I) (FcγRIII:huFcγRIIb) ratio in IgG affinity is important for maximal antibody-mediated effector activity⁵⁶. FIG. 13B shows the data expressed as a ratio of hCSL360 variant affinities for FcγRIII:huFcγRII. All the variants demonstrated increased A/I ratio relative to hCSL360 with ˜2-fold, ˜4-fold and ˜3-fold increase in A/I for hCSL360kif, hCSL360Fc2 and hCSL360Fc3 respectively.

These data confirm that, as expected, the various hCSL360 Fc enhanced variants exhibit increased affinities for FcγR's with greater effects for the activating versus inhibitory FcγR's.

### Discussion

It is shown here that Fc-engineered and defucosylated CSL360 variants demonstrate significantly increased affinities and A/I binding ratio's for FcRγ as well as improved ADCC effector activity in vitro. This result, taken together with the data provided in Examples 1 and 3 demonstrating an important role for effector function activity for therapeutic efficacy of anti-CD123 antibodies in mouse models of AML, strongly suggest that effector function enhanced variant anti-CD123 antibody therapeutics would likely demonstrate improved therapeutic activity for the treatment of AML and other CD123-positive leukemias in human patients.

### Example 5

In this example, the various Fc-enhanced antibodies were tested for enhanced ADCC activity against cell lines engineered to express CD123 as well as human leukemic cell lines that express native CD123. The Fc-enhanced MAb's were also tested using ex vivo ADCC assays against a panel of primary leukemia samples from AML and ALL patients.

### Methods

### Measuring ADCC Using a Lactate Dehydrogenase Release Assay

ADCC was measured using a lactate dehydrogenase (LDH) release assay as described³⁵. LDH is a stable cytosolic enzyme that is released upon cell lysis. LDH released in to the culture medium is measured using a colorimetric assay where LDH converts a specific substrate into a red coloured product. Lysis is measured as LDH released and is directly proportional to the colour formed. Target cells that express CD123 were incubated with varying amounts of anti-CD123 antibodies in the presence of NK cells used as effector cells for ADCC. NK cells were purified from a normal buffy pack using Miltenyi Biotec's NK Isolation Kit (Cat#130-092-657). Cells were incubated for a period of four hours at 37° C. in presence of 5% CO₂. Target cells with no antibody or NK cells were used as spontaneous LDH release (background) controls and target cells lysed with lysis buffer were used as maximal lysis controls. LDH released into the culture media was measured using Promega's CytoTox 96® Non-Radioactive Cytotoxicity Assay Kit according to manufacturers instructions (Cat# G1780).

All other methods are as described in the previous Examples.

### Results

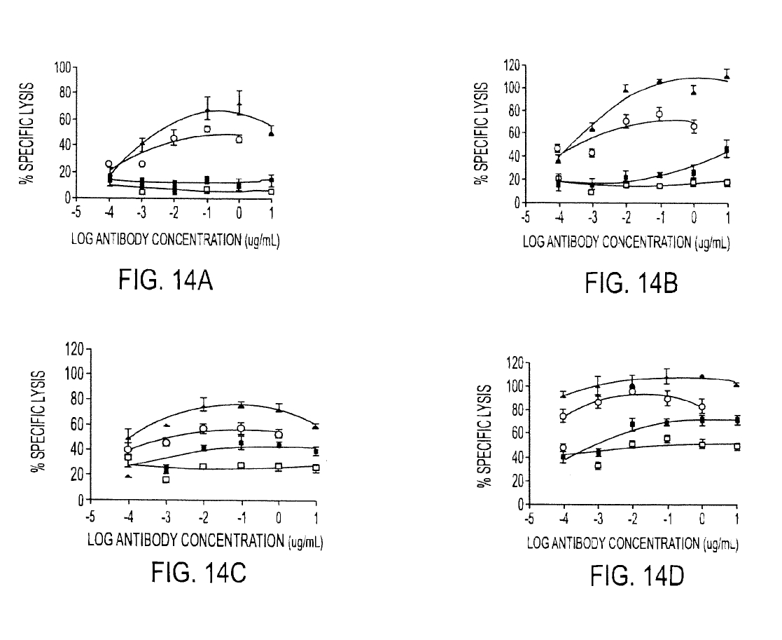

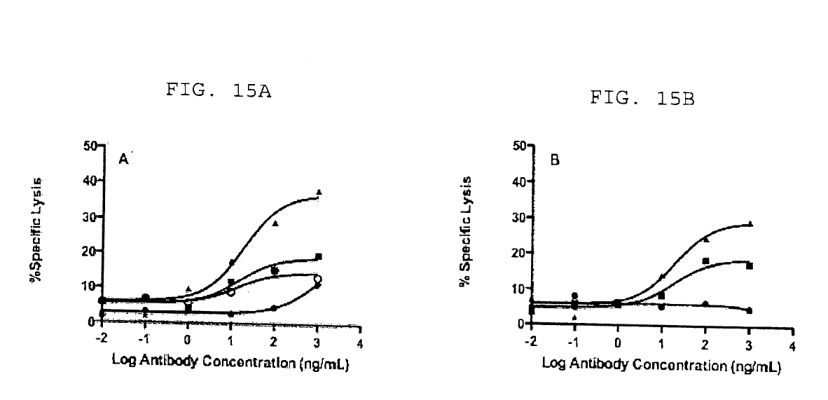

FIG. 14 examines the effects of the various CSL360 derivative antibodies on ADCC activity against human lymphoblastoid Raji cells engineered to express CD123. A stable clone expressing low levels of CD123 (˜4,800 receptors/cell) (Raji-CD123 low) (FIGS. 14a and b) or an independent clone expressing high levels of CD123 (˜24,400 receptors/cell) (Raji-CD123 high) (FIGS. 14c and d) were used for these experiments. Effector to target cell ratios of 25:1 and 50:1 were used. Consistently, hCSL360Fc3 and CSL360kif demonstrated significantly improved ADCC activity against both the Raji-CD123 low and Raji-CD123 high compared to the parent hCSL360 antibody. At the E:T ratio of 50:1 both hCSL360Fc3 and hCSL360kif achieved almost complete lysis of the Raji-CD123 high target cells at low concentrations (˜1 ng/mL) of antibody. Approximately one order of magnitude more antibody was required for equivalent effects in the Raji-CD123 low cells. Interestingly, chimeric CSL360-induced ADCC was marginally more pronounced (albeit at a lower level than the Fc enhanced variants) compared to hCSL360. This may be due to the ˜10-fold decreased affinity for the humanized variant for CD123 binding compared to the chimeric MAb which resulted from the humanization process as discussed earlier.

FIG. 15a shows a repeat of the above experiment this time using TF-1 human leukemic cells which naturally express CD123 as target cells. Once again the hCSL360Fc3 variant showed significantly improved ADCC with hCSL360Fc2 and hCSL360kif, although less potent, also demonstrating increased activity compared to Fc unoptimised hCSL360.

FIG. 15b compares in TF-1 cells the activity of the humanised and affinity optimised anti-CD123 antibody variant 168-26 and its Fc-enhanced derivatives 168-26Fc3 and 168-26Fc2. The data in this Figure demonstrate that Fc engineering improved ADCC activity of the humanised and affinity optimised 168-26 variant similarly to that seen with the humanised only variant (hCSL360).

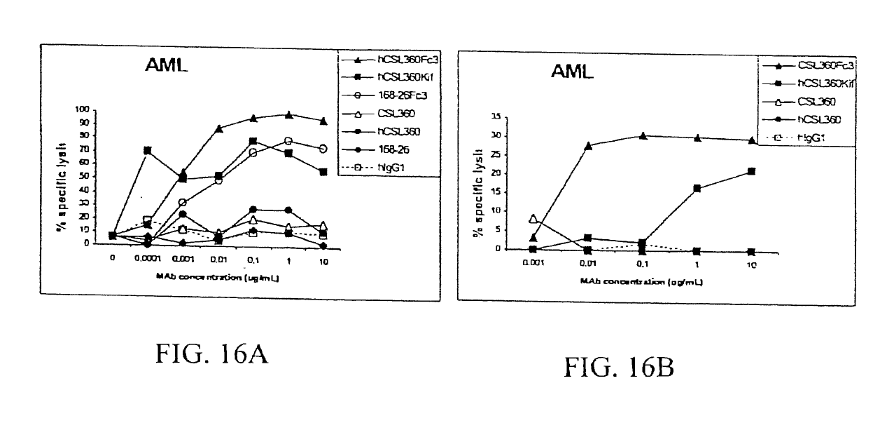

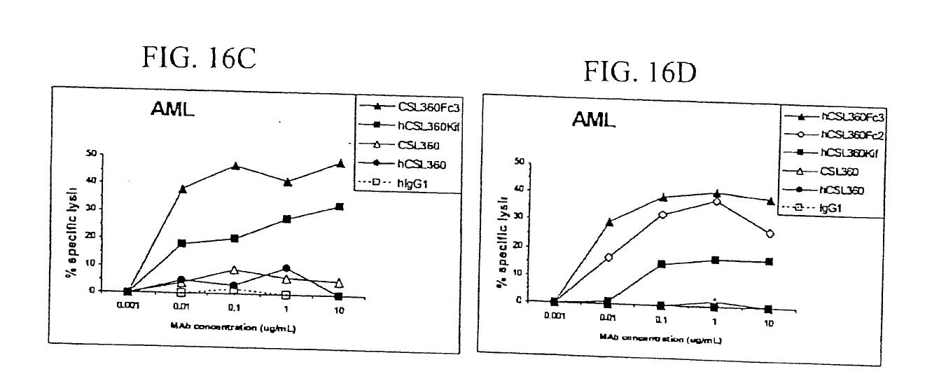

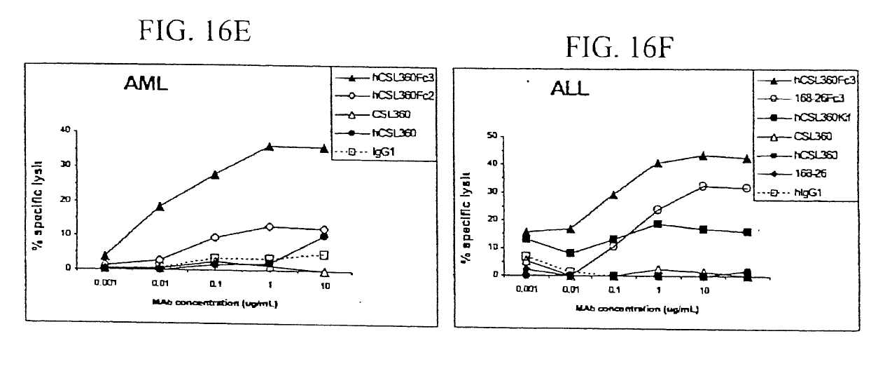

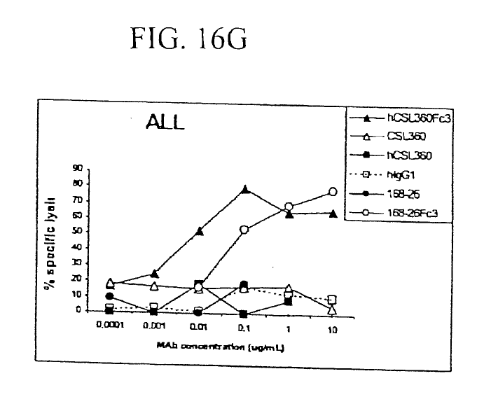

Next, the activity for the various Fc-enhanced hCSL360 variants was compared against a panel of primary leukemic cell samples from 5 AML patients (FIGS. 16 a-e) and 2 ALL patients (FIGS. 16 f-g). The results in these primary patient samples were similar to those obtained using the cell lines with rank order of potency for ADCC activity being hCSL360Fc3≧168-26Fc3>hCSL360Fc2≧hCSL360kif>>CSL360≧168-26≧hCSL360.

Importantly, the Fc-optimised variants consistently induced ADCC in all the primary patient samples tested. All 5 AML and both ALL samples demonstrated significantly higher levels of ADCC by the Fc optimised variants whereas for the variants without Fc optimisation only 3 of the AML samples demonstrated a weak response. Neither ALL sample demonstrated any significant ADCC response to the non Fc optimised variant MAbs.

These data are consistent with the results depicted in Example 2 where CSL360 treatment induced modest ADCC activity in 4/6 AML samples and 0/2 ALL samples assessed by various ADCC methodologies.

### Discussion

The data in Example 5 demonstrate that Fc optimisation of the CD123 MAbs resulted in significant effector function responses against all primary leukemia samples tested in ex vivo assays and represents a significant improvement compared to Fc unoptimised anti-CD123 MAbs.

These findings with ALL tumors that express CD123 are consistent with the notion that other malignancies that express CD123 in addition to AML are also likely to be sensitive to anti-CD123 MAb therapeutics with enhanced Fc effector functions⁵⁷⁻⁶¹.

### Example 6

The results described in Examples 4 and 5 indicate that CSL360 variants with enhanced Fc effector function exhibit increased ADCC activity in vitro against a panel of cell lines engineered to express CD123, human leukemic cell lines which naturally express CD123 and importantly also in ex vivo assays using primary leukemic samples taken from patients with AML or ALL. The ex vivo ADCC data against both AML and ALL patient primary samples is particularly important as testing in this ex vivo setting allows for some estimation of the potential for efficacy in a human disease setting.

In this example, the experiments are extended to test an Fc-engineered variant of CSL360 (168-26Fc3) for therapeutic efficacy in a NOD/SCID mouse xenograft model of human ALL. This is a preclinical model which has been demonstrated to accurately reflect ALL clinical disease and significantly correlates with patient outcome⁶². The clinical relevance of this model is well recognized and is currently an integral part of the National Cancer Institute initiative: the Pediatric Preclinical Testing Program⁶³.

### Methods

Human ALL leukemia cells (ALL-2) derived from a pediatric ALL patient were propagated by intravenous inoculation in female non-obese diabetic (NOD)/scid−/− mice as described previously⁶². This xenograft was derived from the third relapse of a 65 month old female diagnosed with common CD10⁺ B-cell precursor ALL. The patient has since died of her disease and this xenograft is resistant to conventional chemotherapy⁶². Mice were randomized into treatment and control groups of 6-7 mice each to give an approximately equal median leukemic burden in all groups at commencement of treatment. All mice were maintained under barrier conditions and experiments were conducted using protocols and conditions approved by the Committee and the Animal Care and Ethics Committee of the University of New South Wales. Percentages of human CD45-positive (hCD45+) cells were determined as previously described⁶².

The exact log-rank test, as implemented using GraphPad Prism 4.0a, was used to compare event-free survival distributions between treatment and control groups. P values were two-sided and were not adjusted for multiple comparisons given the exploratory nature of the studies.

Treatment commenced on day 34 post transplantation and mice received treatments of 300 μg per 100 μL of antibody dissolved in phosphate-buffered saline. Antibodies were administered by intraperitoneal injection given three times per week (every 2-3 days). Leukemic burden was monitored by weekly tail vein bleed of the mice. Treatment continued until event was reached and was defined as 25% hCD45+ burden in peripheral blood.

### Results

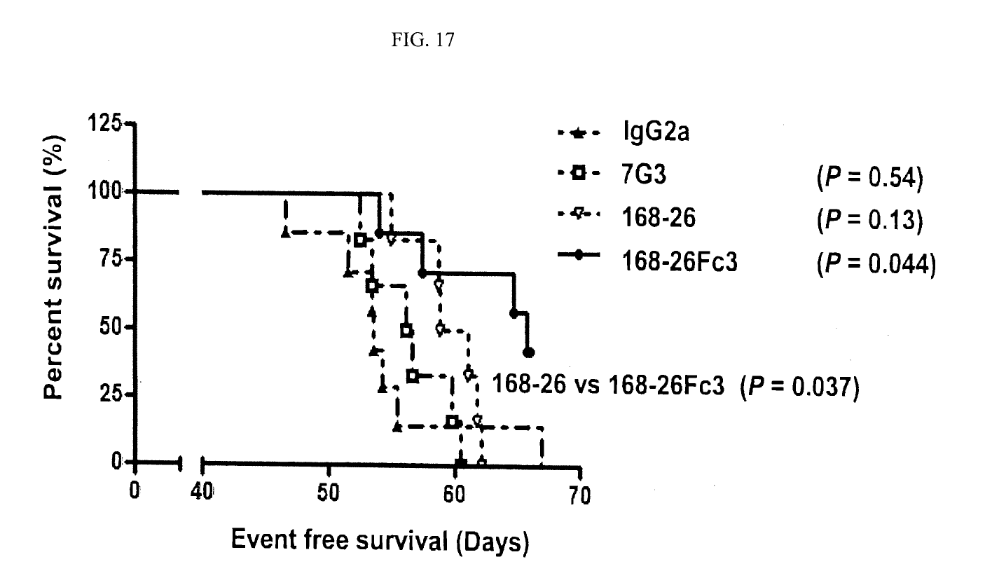

FIG. 17 examines the effect on ALL-engrafted mice for the various antibodies including an irrelevant MAb control (murine IgG2a), murine MAb 7G3, the humanised and affinity optimised variant 168-26 and the latter's Fc-engineered variant 168-26Fc3. The figure depicts Kaplan-Meier curves for event-free survival (EFS) for each of the treatment groups with each vertical line representing an event. Mice treated with control MAb exhibited a median EFS of 53.5 days compared to 56.3, 59.9 and 65.7 days for 7G3, 168-26 and 168-26Fc3 respectively. The results show that 7G3 and 168-26 although delaying the growth of the leukemia by 2.9 and 6.4 days respectively, that the effects were not statistically significant compared to control MAb (P>0.05). 168-26Fc3 exhibited the most profound effect on growth of the ALL with a statistically significant delay in leukemia growth of 12.2 days compared to control treated animals (P=0.044). Importantly, the increased EFS effect of the Fc-engineered variant 168-26Fc3 vs 168-26 (the same MAb without Fc modifications) was statistically significant (P=0.037) with a leukemic growth delay of 5.9 days. This demonstrates that anti-CD123 antibodies with enhanced Fc effector function exhibit improved therapeutic efficacy in vivo.

### CONCLUSION

These data significantly extend those presented in the previous examples in that they demonstrate that anti-CD123 MAbs with enhanced Fc effector function have improved therapeutic efficacy in mice with pre-established leukemia compared to Fc-unmodified MAbs. Importantly, the use of a preclinically validated model of ALL that has been demonstrated to predict the course of human disease⁶² strongly supports that such Fc optimised anti CD123 MAbs may also exhibit improved clinical efficacy in leukemic patients.

### REFERENCES

1. Wang, J. C. & Dick, J. E. Cancer stem cells: lessons from leukemia. Trends Cell Biol (2005).
2. Bonnet, D. & Dick, J. E. Human acute myeloid leukemia is organized as a hierarchy that originates from a primitive hematopoietic cell. Nat Med, 3, 730-737 (1997).
3. Hope, K. J., Jin, L. & Dick, J. E. Acute myeloid leukemia originates from a hierarchy of leukemic stem cell classes that differ in self-renewal capacity. Nat Immunol. 5, 738-743 (2004).
4. Lapidot, T., et al. A cell initiating human acute myeloid leukaemia after transplantation into SCID mice. Nature 367, 645-648 (1994).
5. Guan, Y. & Hogge, D. E. Proliferative status of primitive hematopoietic progenitors from patients with acute myelogenous leukemia (AML). Leukemia 14, 2135-2141 (2000).
6. Guzman, M. L., et al. Nuclear factor-kappaB is constitutively activated in primitive human acute myelogenous leukemia cells. Blood 98, 2301-2307 (2001).
7. Lowenberg, B., Griffin, J. D. & Tallman, M. S. Acute myeloid leukemia and acute promyelocytic leukemia. Hematology Am Soc Hematol Educ Program, 82-101 (2003).
8. van Rhenen, A., et al. High stem cell frequency in acute myeloid leukemia at diagnosis predicts high minimal residual disease and poor survival. Clin Cancer Res 11, 6520-6527 (2005).
9. Morgan, M. A. & Reuter, C. W. Molecularly targeted therapies in myelodysplastic syndromes and acute myeloid leukemias. Ann Hematol 85, 139-163 (2006).
10. Tallman, M. S. New agents for the treatment of acute myeloid leukemia. Best Pract Res Clin Haematol 19, 311-320 (2006).
11. Aribi, A., Ravandi, F. & Giles, F. Novel agents in acute myeloid leukemia. Cancer J. 12, 77-91 (2006).
12. Abutalib, S. A. & Tallman, M. S. Monoclonal antibodies for the treatment of acute myeloid leukemia. Curr Pharm Biotechnol. 7, 343-369 (2006).
13. Stone, R. M. Novel therapeutic agents in acute myeloid leukemia. Exp Hematol. 35, 163-166 (2007).
14. Krug, U., et al. New molecular therapy targets in acute myeloid leukemia. Recent Results Cancer Res. 176, 243-262 (2007).
15. Miyajima, A., Mui, A. L., Ogorochi, T. & Sakamaki, K. Receptors for granulocyte-macrophage colony-stimulating factor, interleukin-3, and interleukin-5. Blood 82, 1960-1974 (1993).
16. Bagley, C. J., Woodcock, J. M., Stomski, F. C. & Lopez, A. F. The structural and functional basis of cytokine receptor activation: lessons from the common beta subunit of the granulocyte-macrophage colony-stimulating factor, interleukin-3 (IL-3), and IL-receptors. Blood 89, 1471-1482 (1997).
17. Munoz, L., et al. Interleukin-3 receptor alpha chain (CD123) is widely expressed in hematologic malignancies. Haematologica 86, 1261-1269 (2001).
18. Sperr, W. R., et al. Human leukaemic stem cells: a novel target of therapy. Eur J Clin Invest. 34 Suppl 2, 31-40 (2004).
19. Graf, M., et al. Expression and prognostic value of hemopoietic cytokine receptors in acute myeloid leukemia (AML): implications for future therapeutical strategies. Eur J Haematol. 72, 89-106 (2004).
20. Florian, S., et al. Detection of molecular targets on the surface of CD34+/CD38− stem cells in various myeloid malignancies. Leuk Lymphoma 47, 207-222 (2006).
21. Testa, U., et al. Elevated expression of IL-3Ralpha in acute myelogenous leukemia is associated with enhanced blast proliferation, increased cellularity, and poor prognosis. Blood 100, 2980-2988 (2002).
22. Riccioni, R., et al. Immunophenotypic features of acute myeloid leukemias overexpressing the interleukin 3 receptor alpha chain. Leuk Lymphoma 45, 1511-1517 (2004).
23. Yalcintepe, L., Frankel, A. E. & Hogge, D. E. Expression of interleukin-3 receptor subunits on defined subpopulations of acute myeloid leukemia blasts predicts the cytotoxicity of diphtheria toxin interleukin-3 fusion protein against malignant progenitors that engraft in immunodeficient mice. Blood 108, 3530-3537 (2006).
24. Hauswirth, A. W., et al. Expression of the target receptor CD33 in CD34+/CD38−/CD123+ AML stem cells. Eur J Clin Invest. 37, 73-82 (2007).
25. Jordan, C. T., et al. The interleukin-3 receptor alpha chain is a unique marker for human acute myelogenous leukemia stem cells. Leukemia 14, 1777-1784 (2000).
26. Budel, L. M., Touw, I. P., Delwel, R., Clark, S. C. & Lowenberg, B. Interleukin-3 and granulocyte-monocyte colony-stimulating factor receptors on human acute myelocytic leukemia cells and relationship to the proliferative response. Blood 74, 565-571 (1989).
27. Salem, M., et al. Maturation of human acute myeloid leukaemia in vitro: the response to five recombinant haematopoietic factors in a serum-free system. Br J Haematol. 71, 363-370 (1989).
28. Delwel, R., et al. Growth regulation of human acute myeloid leukemia: effects of five recombinant hematopoietic factors in a serum-free culture system. Blood 72, 1944-1949 (1988).
29. Miyauchi, J., et al. The effects of three recombinant growth factors, IL-3, GM-CSF, and G-CSF, on the blast cells of acute myeloblastic leukemia maintained in short-term suspension culture. Blood 70, 657-663 (1987).
30. Pebusque, M. J., et al. Recombinant human IL-3 and G-CSF act synergistically in stimulating the growth of acute myeloid leukemia cells. Leukemia 3, 200-205 (1989).
31. Vellenga, E., Ostapovicz, D., O'Rourke, B. & Griffin, J. D. Effects of recombinant IL-3, GM-CSF, and G-CSF on proliferation of leukemic clonogenic cells in short-term and long-term cultures. Leukemia 1, 584-589 (1987).
32. Testa, U., et al. Interleukin-3 receptor in acute leukemia. Leukemia 18, 219-226 (2004).
33. Sun, Q., et al. Monoclonal antibody 7G3 recognizes the N-terminal domain of the human interleukin-3 (IL-3) receptor alpha-chain and functions as a specific IL-3 receptor antagonist. Blood 87, 83-92 (1996).
34. Niwa, R., et al. IgG1 subclass-independent improvement of antibody-dependent cellular cytotoxicity by fucose removal from Asn²⁹⁷-linked oligo saccharides. J. Immunol. Methods, 306, 151-160 (2005).
35. Lazar, G. A., et al. Engineered antibody Fc variants with enhanced effector function. Proc. Natl. Acad. Sci. USA, 103, 4005-4010 (2006).
36. Shields, R. L., et al. Lack of fucose on human IgG1 N-linked oligosaccharide improves binding to human Fc gamma RIII and antibody dependent cellular toxicity. J. Biol. Chem., 277:26733-26740 (2002).
37. Desjarlais et al Optimizing engagement of the immune system by anti-tumor antibodies: an engineer's perspective. Drug Discov Today. 2007 November; 12(21-22):898-910).
38. Mazurier, F., Doedens, M., Gan, O. I. & Dick, J. E. Rapid myeloerythroid repopulation after intrafemoral transplantation of NOD-SCID mice reveals a new class of human stem cells. Nat Med. 9, 959-963 (2003).
39. Lopez, A. F., et al. Recombinant human interleukin-3 stimulation of hematopoiesis in humans: loss of responsiveness with differentiation in the neutrophilic myeloid series. Blood 72, 1797-1804 (1988).
40. Guthridge, M. A., et al. The phosphoserine-585-dependent pathway of the GM-CSF/IL-3/IL-5 receptors mediates hematopoietic cell survival through activation of NF-kappaB and induction of bcl-2. Blood 103, 820-827 (2004).
41. Guthridge, M. A., et al. Site-specific serine phosphorylation of the IL-3 receptor is required for hemopoietic cell survival. Mol Cell 6, 99-108 (2000).
42. Tanaka, T., et al. Selective long-term elimination of natural killer cells in vivo by an anti-interleukin 2 receptor beta chain monoclonal antibody in mice. J Exp Med. 178, 1103-1107 (1993).
43. McKenzie, J. L., Gan, O. I., Doedens, M. & Dick, J. E. Human short-term repopulating stem cells are efficiently detected following intrafemoral transplantation into NOD/SCID recipients depleted of CD122+ cells. Blood 106, 1259-1261 (2005).
44. Dick, J. E. & Lapidot, T. Biology of normal and acute myeloid leukemia stem cells. Int J Hematol. 82, 389-396 (2005).
45. Kollet, O., et al. Rapid and efficient homing of human CD34(+)CD38(−/low)CXCR4(+) stem and progenitor cells to the bone marrow and spleen of NOD/SCID and NOD/SCID/B2m (null) mice. Blood 97, 3283-3291 (2001).
46. Lapidot, T., Dar, A. & Kollet, O. How do stem cells find their way home? Blood 106, 1901-1910 (2005).
47. Hogge, D. E., Feuring-Buske, M., Gerhard, B. & Frankel, A. E. The efficacy of diphtheria-growth factor fusion proteins is enhanced by co-administration of cytosine arabinoside in an immunodeficient mouse model of human acute myeloid leukemia. Leuk Res. 28, 1221-1226 (2004).
48. Feuring-Buske, M., Frankel, A. E., Alexander, R. L., Gerhard, B. & Hogge, D. E. A diphtheria toxin-interleukin 3 fusion protein is cytotoxic to primitive acute myeloid leukemia progenitors but spares normal progenitors. Cancer Res. 62, 1730-1736 (2002).
49. Du, X., Ho, M. & Pastan, I. New immunotoxins targeting CD123, a stem cell antigen on acute myeloid leukemia cells. J Immunother (1997) 30, 607-613 (2007).
50. Jenkins et al., A Cell Type-specific Constitutive Point Mutant of the Common-Subunit of the Human Granulocyte-Macrophage Colony-stimulating Factor (GM-CSF), Interleukin (IL)-3, and IL-5 Receptors Requires the GM-CSF Receptor-Subunit for Activation. J Biol Chem, 1999 274:13, 8669-8677)
51. Fischer, Lars, Olaf Penack, Chiara Gentilini, Axel Nogai, Arne Muessig, Eckhard Thiel and Lutz Uharek. The anti-lymphoma effect of antibody-mediated immunotherapy is based on an increased degranulation of peripheral blood natural killer (NK) cells. Exp Hematol. 34: 753-759 (2006)
52. Neri S, Mariani E, Meneghetti A, Cattini L and Facchini A. Calcein-Acetoxymethyl cytotoxicity assay: standardisation of a method allowing additional analyses on recovered effector cells and supernatants. Clinical and Diagnostic Lab Immunol. 8:1131-1135 (2001)
53. Zhou et al, Development of a simple and rapid method for producing non-fucosylated oligomannose containing antibodies with increased effector function. Biotechnol Bioeng. 2008 99(3):652-65)
54. Tan, P., et al, “Superhumanized” Antibodies: reduction of immunogenic potential by complementarity-determining region grafting with human germline sequences: application to an anti-CD28. 2002 J Immunol. 169, 1119-1125)
55. Kopsidas, G., et al, RNA mutagenesis yields highly diverse mRNA libraries for in vitro protein evolution. 2007 BMC Biotechnol. 7, 18)
56. Nimmerjahn and & Ravetch, Divergent immunoglobulin g subclass activity through selective Fc receptor binding. Science 2005 310(5753):1510-2).
57. Feuillard et al, Clinical and biologic features of CD4(+)CD56(+) malignancies Blood 2002 99(5):1556-63
58. Muñoz et al, Interleukin-3 receptor alpha chain (CD123) is widely expressed in hematologic malignancies Haematologica 2001 86(12):1261-9
59. Lhermitte et al, Most immature T-ALLs express Ra-IL3 (CD123): possible target for DT-IL3 therapy. Leukemia 2006 20(10): 1908-10
60. Aldinucci et al, The role of interleukin-3 in classical Hodgkin's disease. Leuk Lymphoma 2005 46(3):303-11
61. Jiang et al, Autocrine production and action of IL-3 and granulocyte colony-stimulating factor in chronic myeloid leukemia. Proc Natl Acad Sci USA. 1999 26; 96(22):12804-9)
62. Liem et al, Characterization of childhood acute lymphoblastic leukemia xenograft models for the preclinical evaluation of new therapies Blood 2004; 103(10):3905-14)
63. Houghton et al, Testing of New Agents in Childhood Cancer Preclinical Models. Clinical Cancer Research. 2002; 8:3646-3657)

1.-23. (canceled)

24. A method for inhibition of leukemic stem cells expressing IL-3Rα (CD123), which comprises contacting said cells with an antigen binding molecule comprising a Fc region or a modified Fc region having enhanced Fc effector function, wherein said antigen binding molecule binds selectively to IL-3Rα (CD123).

25. A method for the treatment of a hematologic cancer condition in a patient, which comprises administration to the patient of an effective amount of an antigen binding molecule comprising a Fc region or a modified Fc region having enhanced Fc effector function, wherein said antigen binding molecule binds selectively to IL-3Rα (CD123).

26. The method of claim 25 wherein the patient is a human.

27. The method of claim 24, wherein the antigen binding molecule is a monoclonal antibody or antibody fragment comprising a Fc region.

28. The method of claim 24, wherein the antigen binding molecule is a monoclonal antibody or antibody fragment comprising a modified Fc region having enhanced Fe effector function.

29. The method of claim 28, wherein the modification in the Fc region of the antibody or antibody fragment comprises substitution of at least one amino acid, preferably two or three amino acids, in the Fc region to enhance the interaction of the Fc region with relevant Fc receptors and complement.

30. The method of claim 28, wherein the antibody or antibody fragment comprising a modified Fc region is a defucosylated antibody or antibody fragment.

31. The method of claim 28, wherein the modification in the Fc region of the antibody or antibody fragment comprises modification of an oligosaccharide attached at the conserved Asn²⁹⁷ in the Fc region.

32. The method of claim 27, wherein the antigen binding molecule is a chimeric, humanized or human monoclonal antibody or antibody fragment.

33. The method of claim 32, wherein the antigen binding molecule is a chimeric antibody or antibody fragment comprising light variable and heavy variable regions of a mouse anti-CD123 monoclonal antibody grafted onto a human constant region.

34. The method of claim 32, wherein the antigen binding molecule is a humanized antibody or antibody fragment comprising complementarity-determining regions (CDRs) of a mouse anti-CD123 monoclonal antibody grafted on a human framework region.

35. The method of claim 25, wherein said hematologic cancer condition is leukemia or a malignant lymphoproliferative disorder.

36. The method of claim 35, wherein said leukemia is selected from the group consisting of acute myelogenous leukemia, chronic myelogenous leukemia, acute lymphoid leukemia, chronic lymphoid leukemia, and myelodysplastic syndrome.

37. The method of claim 35, wherein said malignant lymphoproliferative disorder is lymphoma.

38. The method of claim 37, wherein said lymphoma is selected from the group consisting of multiple myeloma, non-Hodgkin's lymphoma, Burkitt's lymphoma, and small cell- and large cell-follicular lymphoma.

39. The method of claim 25, further comprising administration to said patient of a chemotherapeutic agent.

40. The method of claim 39, wherein administration of the chemotherapeutic agent is prior to, simultaneous with, or subsequent to, administration of the antigen binding molecule.

41. The method of claim 39, wherein said chemotherapeutic agent is a cytotoxic agent selected from the group consisting of:
(a) Mustard gas derivatives: Mechlorethamine, Cyclophosphamide, Chlorambucil, Melphalan, and Ifosfamide
(b) Ethylenimines: Thiotepa and Hexamethylmelamine
(c) Alkylsulfonates: Busulfan
(d) Hydrazines and triazines: Althretamine, Procarbazine, Dacarbazine and Temozolomide
(e) Nitrosureas: Carmustine, Lomustine and Streptozocin
(f) Metal salts: Carboplatin, Cisplatin, and Oxaliplatin
(g) Vinca alkaloids: Vincristine, Vinblastine and Vinorelbine
(h) Taxanes: Paclitaxel and Docetaxel
(i) Podophyllotoxins: Etoposide and Tenisopide.
(j) Camptothecan analogs: Irinotecan and Topotecan
(k) Anthracyclines: Doxorubicin, Daunorubicin, Epirubicin, Mitoxantrone and Idarubicin
(l) Chromomycins: Dactinomycin and Plicamycin
(m) Miscellaneous antitumor antibiotics: Mitomycin and Bleomycin
(n) Folic acid antagonists: Methotrexate
(o) Pyrimidine antagonists: 5-Fluorouracil, Foxuridine, Cytarabine, Capecitabine, and Gemcitabine
(p) Purine antagonists: 6-Mercaptopurine and 6-Thioguanine
(q) Adenosine deaminase inhibitors: Cladribine, Fludarabine, Nelarabine and Pentostatin
(r) Topoisomerase I inhibitors: Ironotecan and Topotecan
(s) Topoisomerase II inhibitors: Amsacrine, Etoposide, Etoposide phosphate and Teniposide
(t) Ribonucleotide reductase inhibitors: Hydroxyurea
(u) Adrenocortical steroid inhibitors: Mitotane
(v) Enzymes: Asparaginase and Pegaspargase
(w) Antimicrotubule agents: Estramustine
(x) Retinoids: Bexarotene, Isotretinoin and Tretinoin (ATRA).

42. The method of claim 41, wherein said cytotoxic agent is Cytarabine.


SEQUENCE LISTING (2 sequences)

SEQ ID NO: 1 - DNA, 24 bp, Artificial Sequence
  Description of Artificial Sequence Synthetic primer
aataacccttgaccaggcatccta

SEQ ID NO: 2 - DNA, 24 bp, Artificial Sequence
  Description of Artificial Sequence Synthetic primer
ctgaggcacctccagatgttaact